(19) United States
(12) Patent Application Publication
(10) Pub. No.: US 2007/0275046 A1
(43) Pub. Date: Nov. 29, 2007

(54) Noval Surfactants and Applications Thereof

(75) Inventors: Bernard Pucci, Molleges (FR); Ange Polidori, Avignon (FR); Nicolas Michel, Avignon (FR); Anne-Sylvie Fabiano, Althen Les Paluds (FR); Christiane Contino-Pepin, Althen Des Paluds (FR); Jean-Pierre Salles, Eguilles (FR)

Correspondence Address: MERCHANT & GOULD PC, P.O. BOX 2903, MINNEAPOLIS, MN, 55402-0903, US

(73) Assignees: TS PHARMA, Eguilles (FR); UNIVERSITE D'AVIGNON ET DES PAYS DU VAUCLUSE, Avignon Cedex (FR)

(21) Appl. No.: 10/576,767

(22) PCT Filed: Oct. 19, 2004
(86) PCT No.: PCT/FR04/02667

§ 371 Date: May 22, 2007

(30) Foreign Application Priority Data
Oct. 23, 2003 (FR) 0312390
May 14, 2004 (FR) 0405276

(51) Int. Cl. A61K 31/221; C07C 323/60

(52) U.S. Cl. 424/450; 514/784; 554/37; 554/85

(57) ABSTRACT

The invention relates to novel surfactant compounds and the use thereof for preparing metastable supramolecular systems or nanoparticles. Said nanoparticles may be used as vectors for active ingredients, in particular therapeutic active ingredients.

The present invention relates to novel surfactants of telomer type, and to the use thereof for preparing metastable supramolecular systems. These metastable supramolecular systems or nanoparticles may be liposomes, or micellar systems. The invention also relates to the atypical liposomes and nanoparticles obtained from these surfactants and to the use thereof as vectors for active ingredients, in particular for therapeutic active ingredients.

Some amphiphilic molecules, natural phospholipids, have the property of associating in water by forming spherically shaped metastable supramolecular organizations called liposomes which contain an inner aqueous compartment. These liposomes are capable of containing therapeutic active agents within this inner compartment and can thus be used to transport these active agents to target cells or tissues. The study of these particulate vectors has been the subject of an abundant literature in which the problems and also the potentialities of the use thereof have been widely dealt with (Barenholz, Curr. Opin. In Coll. and Int. Sci. 6 (2001) 66-77).

However, the use of liposomes for transporting therapeutic active ingredients has some major drawbacks:

These nanostructures generally have a relatively low stability over time, since, in the medium in which they are dispersed, they fuse to form larger objects which subsequently rapidly precipitate. This behavior greatly limits their conservation and storage capacity.

The biological stability of these vectors, i.e. their retention time in the bloodstream, is closely associated with their size, which must be less than 200 nm so as to allow them to reach a potential target (Nagayasu et al, Adv. Drug Del. Rev. 40 (1999) 75-87). However, the candidates most effective from this point of view: unilamellar liposomes of small sizes, have the drawback of having an encapsulated medicinal product/lipid ratio that is lower than unilamellar liposomes of large sizes (Vernuri et al., Pharm. Acta Helv. 70 (1995) 95-111). The lipids forming these nanostructures generally have a high production cost. The low capacity for encapsulation of active ingredients of some of these liposomes then represents an economic problem.

Finally, it is essential to be able to provide a vector which can release its contents gradually and continuously. Such properties require the use of highly organized and impermeable membranes which consist of complex and expensive lipid formulations.

Despite these difficulties, a certain number of liposome-based pharmaceutical preparations are currently available on the market or are in the clinical phase. The most appreciable advantage of these preparations is their excellent tolerance with respect to the use of a free active ingredient. Their effectiveness at an equivalent dose is, however, barely greater. This is the case of the liposome-encapsulated amphotericin B (Ambisome®). The encapsulation thereof in lipid formulations considerably increases its therapeutic index (Andres et al., Rev. Méd. Interne, 22 (2001) 141-150).

In order to decrease the rapid elimination thereof by the reticuloendothelial system, systems for protecting the liposomes have been set up. The most effective consists in using phospholipids substituted with polyethylene glycols having a molecular mass of between 1000 and 5000 in a proportion of 5 to 10% of the total mixture of phospholipids. The “invisible” liposomes, referred to as stealth liposomes, thus formed (sold under the trademark Stealth liposomes®) have blood retention times that are longer than conventional liposomes (45 h against a few minutes to a few hours). The increase in blood circulation time of these liposomes promotes their accumulation in cancerous tissues which are particularly irrigated, and their use for the transport of anticancer compounds is particularly suitable (Gabizon et al., Cancer Res. 54 (1994) 987-992). A formulation of Stealth liposomes® based on dauxorubicin (Doxil®, Alza Corp.) is currently marketed for combating Kaposi's syndrome. However, certain formulations consisting of very small (50 nm) liposomes can also be considered to be long-circulation preparations. This is the case of the daunorubicin anticancer formulation marketed by NeXstar (DaunoXome®).

In order to ensure the mechanical stability of the liposomes whether during their storage or their use in vivo, several strategies involving the use of polymers can be envisioned:

Polymerization of the surfactants constituting the membrane of the liposome after its formation (Bader et al., Adv. Polym. Sci. 64 (1985) 1-62) and Hotz et al., Adv. Mater. 10 (1998) 1387-1390).

The interaction of amphiphilic or nonamphiphilic ionic polymers at the surface of the outer membrane of the liposomes (Hayashi et al., Biochim. Biophys. Acta, 1280 (1996) 120-126, Ishihara et al., Coll. and Surf., B: Biointerfaces 25 (2002) 325-333).

Finally, the polymerization of a hydrophilic monomer inside the internal aqueous cavity of the liposome is a method which has been studied very little and has been described briefly by Torchilin et al. (Makromol. Chem., Rapid communication, 8 (1987) 457-460). It has been used as a tool for the production of a polymerized molecular footprint in an American patent (Perrot et al., U.S. Pat. No. 6,217,901, 17 Apr. 2001).

The essential limitation associated with the use of polymers for stabilizing liposomes is the potential toxicity induced by their accumulation in the lysosome or by the nonpolymerized hydrophilic monomer's own toxicity (in the case of acrylamide). In order to limit this phenomenon, it is essential to use low molecular weight polymers that are more readily biodegradable.

The use of micelles stabilized using polymerized amphiphilic compounds combining hydrophilic and hydrophobic blocks for the transport of therapeutic active agents that are relatively insoluble in water has been the subject of abundant research studies (G.S. Kwon et al., Adv. Drug. Deliver. Rev., 16 (1995) 295-309, M. Jones et al., Eur. J. Pharm. Biopharm., 48 (1999) 101-111, V. P. Torchilin, J. Control. Release, 73 (2001) 137-172). These vectorization systems make it possible in particular to transport and solubilize a certain number of anticancer active agents, particularly polycyclic derivatives. As a general rule, the latter exhibit a very low bioavailability when administered orally and the intravenous injection thereof leads, due to aggregation, to embolisms in blood vessels and to a local toxicity due to solid deposits (A. N. Lukyanov et al, Adv. Drug. Deliver. Rev., (2004) available on the Internet, Science Direct). The use of liposomes, of microemulsions or of cyclodextrins is a promising solution, but still exhibits too many limitations, in particular too great a variability in solubilization of these relatively insoluble active agents, which depends to a large extent on their structure. The development of small polymeric micellar systems therefore represents an advantageous alternative to these technologies, in which we were interested.

Due to a particularly low CMC, the polymeric surfactants constituting these micelles confer on them a particularly high thermodynamic stability and a very high capacity for retention of the encapsulated active agents. The very small size of these nanoparticles (less than 100 nm) gives them an excellent stability in vivo and also a passive targeting of particularly well-irrigated tumor sites.

Active targeting of these vectors can be carried out by coating their surface with target molecules such as antibodies, peptides, lectines, sugars, hormones or specific synthetic compounds.

The literature mentions a large number of polymers that are amphiphilic in nature. These are generally diblock-type polymers consisting of various hydrophilic and hydrophobic monomers (M. Jones et al, Eur. J. Pharm. Biopharm., 48 (1999) 101-111, V. P. Torchilin, J. Control. Release, 73 (2001) 137-172). Other amphiphilic agents derived from phospholipids and from polymers of polyethylene glycol and of polyvinylpyrrolidone have also been studied (A. N. Lukyanov et al, Adv. Drug Deliver. Rev., (2004) available on the Internet, Science Direct).

A first objective of the present invention is the development of nanoparticle vectors that have a very low production cost and have the ability to transport, inside their inner aqueous cavity, a very large family of hydrophilic active agents. The nanoparticle vectors of the invention allow the encapsulation, the retention and the release of substances that can be metered. The applications targeted include the transport of active ingredients, in particular of therapeutic active ingredients, the epidermal delivery of cosmetic substances, and medical diagnosis; in particular, the transport of anticancer active agents, of active agents for vaccine-based use, of genetic material, of enzymes, of hormones, of vitamins, of sugars, of proteins and peptides, of lipids, or of organic and inorganic molecules.

This objective has been achieved by virtue of the design and the synthesis of novel surfactants which make it possible to prepare nanoparticle vectors or liposomes that have advantageous properties compared with the liposomes of the prior art.

A second objective of the present invention is the development of nanoparticle vectors that have a very low production cost and have the ability to transport, inside their hydrophobic cavity, or their lipid lamella, a very large family of hydrophobic active agents. The nanoparticle vectors of the invention allow the encapsulation, the retention and the release of substances that can be metered. The applications targeted include the transport of active ingredients, in particular of therapeutic active ingredients, the epidermal delivery of cosmetic substances, and medical diagnosis; in particular, the transport of anticancer active agents, of active agents for vaccine-based use, of genetic material, of enzymes, of hormones, of vitamins, of sugars, of proteins and peptides, of lipids, or of organic and inorganic molecules.

This objective has been achieved by virtue of the design and the synthesis of novel telomer-type surfactants which make it possible to prepare micelles and ellipsoidal nanoparticles or liposomes that have advantageous properties compared with the polymeric micelles of the prior art.

A subject of the present invention is the compounds corresponding to formula (I): 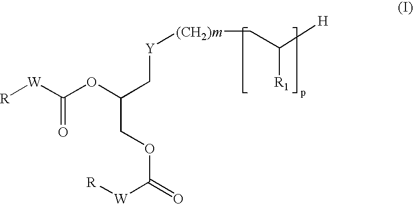
in which:
Y represents a sulfur atom or a group 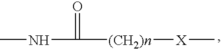
   X being chosen from S and CH₂ groups, n is an integer ranging from 0 to 10, such as, for example, 0, 1, 2, 3, 4, 5 or 6;
m is an integer ranging from 0 to 9, such as, for example, 0, 1, 2, 3, 4, 5 or 6;
   and, when X=CH₂, then 0<m+n<6;
   W represents an —NH—group or a —CH₂— group;
   p represents an integer ranging from 1 to 50;
   R₁ represents a group chosen from the following radicals: 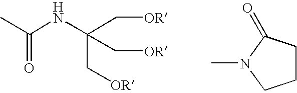
      in which R′ represents H or a hydrophilic group, such as, for example, a C₄-C₂₄ polyhydroxylated hydrocarbon-based compound; in particular R′ can be chosen from sugars, such as, for example, galactose, glucose, mannose or sialic acid, linked via its anomeric carbon;
   R represents a group chosen from: C₄-C₂₄ hydrocarbon-based radicals; C₄-C₂₄ fluorinated hydrocarbon-based radicals; C₄-C₂₄ thioalkyl radicals.

The group R can in particular be chosen from the following radicals:
the thiooctyl radical,
C₄-C₂₄ hydrocarbon-based radicals, such as n-butyl, tert-butyl, isobutyl, n-pentyl, isopentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl or the phytyl radical (CH₃[CH (CH₃) (CH₂)₃]₃CH (CH₃) CH₂CH₂) ),
C₄-C₂₄ fluorinated hydrocarbon-based radicals, such as those corresponding to the formula —(CH₂)ₜ—(CF₂)ᵣF, in which r and t represent two integers with: 24≧r+t≧4, such as, for example:

—(CF₂)₄F; —(CF₂)₅F; —(CF₂)₆F; —(CF₂)₇F; —(CF₂)₈F; —(CF₂)₉F; —(CF₂)₁₀F; —(CF₂)₁₁F; —(CF₂)₁₂F; —(CF₂)₁₃F; —(CF₂)₁₄F; —CH₂—(CF₂)₃F; —CH₂—(CF₂)₄F; —CH₂—(CF₂)₅F; —CH₂—(CF₂)₆F; —CH₂—(CF₂)₇F; —CH₂—(CF₂)₈F; —CH₂—(CF₂)₉F; —CH₂—(CF₂)₁₀F; —CH₂—(CF₂)₁₁F; —CH₂—(CF₂)₁₂F; —CH₂—(CF₂)₉F; —(CH₂—(CF₂)₂F; —(CH₂)₂—(CF₂)₃F; —(CH₂)₂—(CF₂)₄F; —(CH₂)₂—(CF₂)₅F; —(CH₂)₂—(CF₂)₆F; —(CH₂)₂—(CF₂)₇F; —(CH₂)₂—(CF₂)₈F; —(CH₂)₂—(CF₂)₉F; —(CH₂)₂—(CF₂)₁₀F; —(CH₂)₂—(CF₂)₁₁F; —(CH₂)₂—(CF₂)₁₂F; —(CH₂)₃—(CF₂)₁F; —(CH₂)₁₃—(CF₂) F; —(CH₂)₄ (CF₂)₆F; —(CH₂)₄(CF₂)₈F; —(CH₂)₄(CF₂)₁₀F; —(CH₂)₁₀(CF₂)₆F; —(CH₂)₁₀(CF₂)₈F; —(CH₂)₁₀(CF₂)₁₀F, etc.

According to a first preferred variant, a subject of the present invention is the compounds corresponding to formula (IA): 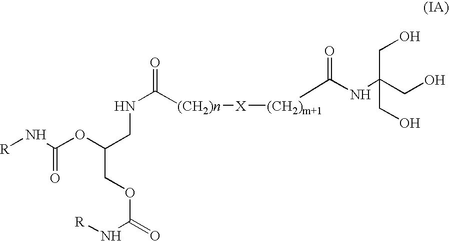
in which:
X represents a sulfur atom S or a —CH₂— group;
n is an integer ranging from 0 to 10, such as, for example, 0, 1, 2, 3, 4, 5 or 6;
m is an integer ranging from 0 to 9, such as, for example, 0, 1, 2, 3, 4, 5 or 6;
when X=CH₂, then 0<m+n<6;
R represents a group chosen from: C₄-C₂₄ hydrocarbon-based radicals; C₄-C₂₄ fluorinated hydrocarbon-based radicals; C₄-C₂₄ thioalkyl radicals.

The preferred R chains are those which contribute to giving the surfactant of formula (I) a phase transition temperature of greater than 37° C. In fact, when such surfactants are used for the production of liposomes, these surfactants, which have a crystalline structure at physiological temperature, give the liposome membrane a greater rigidity and a higher degree of retention of the solutes encapsulated in the inner aqueous compartment. Preferably, R represents a C₁₂-C₂₄ hydrocarbon-based chain or a C₈-C₂₄ fluorinated hydrocarbon-based chain.

Preferably, one or more of the following conditions are met: X=S; n=2, m=1.

The preferred compounds of formula (IA) are those corresponding to formula A in which R has the same definition as above, n=2, X=S, m=1:

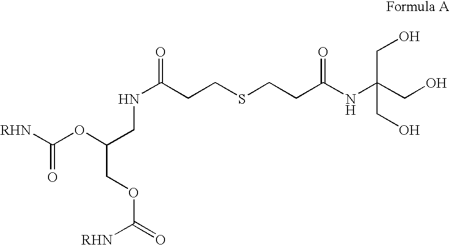

Among these compounds, a particularly preferred compound is Al represented below:

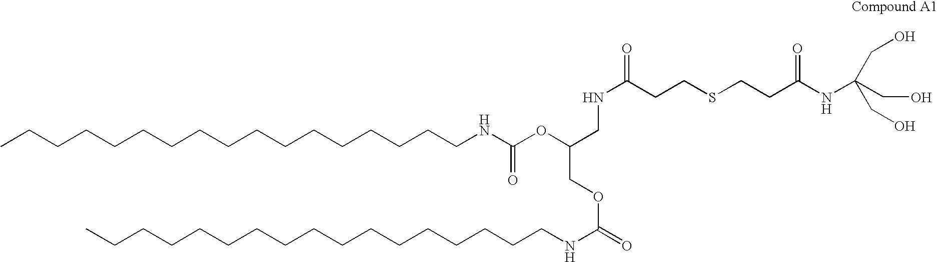

The synthesis of the molecules of formula (I) can be carried out simply, using conventional methods of organic synthesis. Several examples of synthesis are illustrated in the experimental section.

Another subject of the invention consists of the use of the molecules of formula (I), advantageously of the molecules of formula (IA), for the production of liposomes. The walls of the liposomes of the prior art generally consist of phospholipids.

The liposomes are produced from the surfactants of formula (I), preferably of formula (IA), very easily by the film method (Liposomes, a practical approach, R.R.C. New, Ed., Oxford University Press, New York, 1990). This process can be summarized in the following way:

A solution of surfactant (I) or (IA) dissolved in methanol or chloroform is slowly evaporated in a round-bottomed flask in order to form a thin film on the wall of the round-bottomed flask. Distilled water at 65° C. is added in order to rehydrate the film, at a concentration of 2.5 mg/ml. The solution obtained is subsequently subjected to ultrasound for 30 minutes in a sonication bath at a temperature above the phase transition temperature of the dispersed surfactant, until a bluish translucent solution is obtained. For the latter step, it is also possible to replace the sonication with repeated extrusion of the solution through two polycarbonate filters with a porosity of 200 nm, mounted in series. A double treatment of sonication and extrusion can also be envisioned.

Other conventional methods for preparing liposomes can be used for the preparation of the liposomes of the invention. To this effect, reference may be made to S. Vernuri and C. T. Rhodes, Pharmaceutica Acta Helvetiae 70, (1995), 95-111.

Surprisingly, the formation of vesicles with an elongated shape is observed, which vesicles are denoted tubular vesicles due to their size, which is of the order of a few tens of nanometers, and to their shape, which resembles that of a tube closed at both its ends.

The size and the mechanical stability over time of the particles obtained in the solution were measured after filtration by dynamic light diffraction (High Performance Particle Sizer, Malvern). The nature of the particles obtained was studied by transmission electron microscopy after negative staining of the sample or after freeze-fracture (FIG. 4).

The measurements of shape and of size established by viewing the electron micrograph made it possible to demonstrate the formation of liposomes with an elongated shape, called tubular vesicles, closed up at their ends, the average cross section of which is between 20 and 80 nm and the average length of which is between 200 and 500 nm (FIG. 3). The freeze-fracture analyses confirmed the formation of these tubular vesicles and their morphological characteristics, namely the presence of an inner aqueous cavity isolated from the outside medium (FIG. 5).

For a given surfactant of formula (IA), the size of the particles is substantially homogeneous: it varies within a value range of ±10%, preferably of ±5%, around a central value for length and for diameter.

These tubular vesicles have a relatively high stability since no change in particle size is observed after a year of storage, whereas liposomes formed from egg yolk phosphatidylcholine show a change after only 5 days of storage.

The demonstration of the inner aqueous cavity has been proved indirectly by spectrofluorimetric measurements of encapsulation and release kinetics for a hydrophilic fluorescent probe, carboxyfluorescein. The measurements clearly show slower release kinetics for the fluorescent probe compared with a conventional encapsulation in a mixture of egg yolk phosphatidylcholine (FIG. 1).

We have been able to demonstrate that the intermolecular hydrogen bonds between the surfactants constituting the tubular vesicles are the cause of their morphology and of their specific stability. In fact, the alcohol functions of tris(hydroxymethyl)-aminomethane and also the carbamate functions have the property of being able to generate a network of hydrogen bonds between the surfactants constituting the membrane, thus stabilizing the tubular vesicles. It has been possible to demonstrate this by liquid-phase Fourier transform infrared spectroscopy (in CCl₄). An increase in intensity of the band at 1691 cm⁻¹, characteristic of the carbonyl functions linked to a hydrogen atom, is in fact noted on the spectrum when the concentration of surfactant in the solution increases to the detriment of its unbound homolog (FIG. 2).

It should be noted that the substitution in the surfactant of formula (IA), of the carbamate bonds with ester bonds between the fatty chains and the glycerol unit, as illustrated in structure B, leads to the formation, in water, of liposomes with conventional structures, the mechanical stability of which is only a few hours, thus confirming the hypothesis put forward that the tubular vesicles are stabilized and organized due to the establishment of specific hydrogen bonds (FIG. 6).

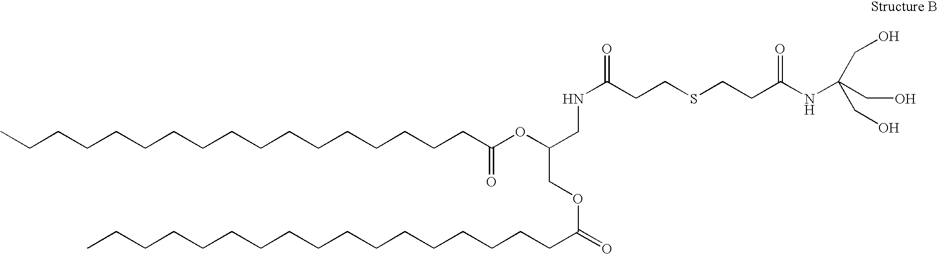

A subject of the invention is also liposomes, or aqueous dispersions of vesicles, characterized in that they contain one or more compounds of formula (I), advantageously of formula (IA), as constituents of their walls.

These liposomes exhibit original structural characteristics which confer on them unexpected properties, in particular improved stability compared with the liposomes of the prior art. The liposomes of the invention have also shown an ability to release an active ingredient over a longer period of time compared with the liposomes of the prior art.

According to a second preferred variant, the subject of the invention is the compounds corresponding to formula (IB): 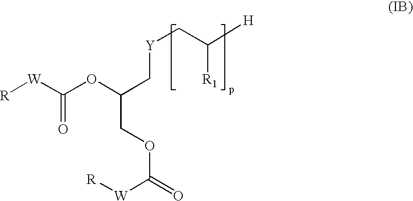
in which:
Y represents a sulfur atom or the —NH—CO—CH₂CH₂S— group;
W represents an —NH— group or a —CH₂— group;
p represents an integer ranging from 1 to 50;
R₁ represents a group chosen from the following radicals: 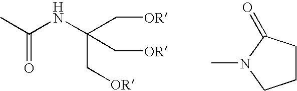
   in which R′ represents H or a hydrophilic group, such as, for example, a C₄-C₂₄ polyhydroxylated hydrocarbon-based compound. In particular, R′ can be chosen from sugars, such as, for example galactose, glucose, mannose or sialic acid, linked via its anomeric carbon.

R represents a group chosen from: C₄-C₂₄ hydrocarbon-based radicals; C₄-C₂₄ fluorinated hydrocarbon-based radicals; C₄-C₂₄ thioalkyl radicals.

The preferred R chains are those which contribute to giving the surfactants of formula (IB) a critical micellar concentration (CMC) of less than 10⁻⁵ M. A low CMC in fact gives the nanoparticle a greater thermodynamic stability and also a greater capacity for retention of the solutes encapsulated in the inner hydrophobic compartment. Preferably, R represents a C₁₂-C₂₄ hydrocarbon-based chain or a C₈-C₂₄ fluorinated hydrocarbon-based chain.

The synthesis of the molecules of formula (IB) can be carried out simply, using conventional methods of organic synthesis. Several examples of synthesis are illustrated in the experimental section.

In summary, when the molecule of formula (IB) is synthesized with an R group of thioalkyl type, said molecule is used as a transfer agent in a telomerization reaction in the presence of the hydrophilic polymerizable reactant (of the type tris(hydroxymethyl)acrylamidomethane or its derivatives or vinylpyrrolidone) and of a free-radical initiator such as α,α′-azobisbutyronitrile (AIBN) in solution in methanol, THF or acetonitrile brought to boiling point. The initial proportion of polymerizable monomer and transfer agent makes it possible to control the degree of polymerization of the telomer and therefore the solubility of the product. The latter are obtained by precipitation from ether.

The preferred compounds of formula (IB) are those for which Y represents S.

Another preferred variant is that in which p represents an integer ranging from 5 to 15.

Even more advantageously, preference is given to the compounds corresponding to formula C below in which R has the same definition as above, p represents an integer ranging from 5 to 15, and W=CH₂:

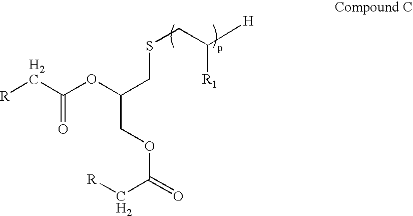

Among these compounds, a particularly preferred compound is Cl represented below:

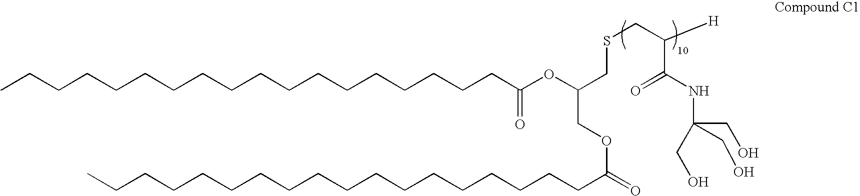

Another subject of the invention consists of the use of the compounds of formula (I), advantageously of the compounds of formula (IB), for the preparation of nanoparticles with a hydrophobic cavity, and the nanoparticles thus obtained. The particles are produced from the surfactants of formula (I) or (IB) very readily by the film method which is well known to those skilled in the art and which is described in the work Liposomes, a practical approach, R.R.C. New, Ed., Oxford University Press, New York, 1990. The process is carried out as disclosed above for the compounds of formula (IA).

Surprisingly, using the compounds of general formula (IB), in which the value of p is less than 15, and when R represents a hydrocarbon-based chain comprising at least 12 carbon atoms, the formation of nanoparticles with an elongated shape, denoted ellipsoids due to their rice-grain shape (FIG. 7a), is observed.

Study of the Ellipsoid Particles Obtained Using the Compounds of Structure (IB)

In fact, using the derivatives of general formula (IB), when p is between 5 and 15, the formation of original particles, the shape of which evokes a rice grain and the size of which decreases when p increases, is observed (FIG. 8). When P is greater than 15, the size of the objects obtained is less than 10 nm, and the nature of the aggregates formed is essentially micellar.

The size and the mechanical stability over time of the particles obtained in the solution were measured after dynamic light diffraction filtration (High Performance Particle Sizer, Malvern). The nature of the particles obtained was studied by transmission electron microscopy after negative staining of the sample or after freeze-fracture. The critical aggregation concentration of these surfactants was determined by tensiometry or by spectrofluorimetry using the fluorescent label method. Furthermore, the Wilhelmy tensiometry technique made it possible to determine the maximum surface tensions and the area of the polar head at the water-air interface (FIG. 8). These compounds exhibit relatively low CMCs of the order of 10⁻⁵ M. The CMC of these surfactants virtually does not change as a function of p (FIG. 8). In order to cause this value to decrease or increase, it is necessary to, respectively, increase or decrease the length of the hydrocarbon-based chains. The phase transition temperature of these surfactants was determined by polarization spectrofluorimetry and by light scattering (FIG. 9). The phase transition temperature virtually does not change, for constant chain lengths when p increases (41° C.<Tm<44° C.).

The hydrodynamic diameter (D_{H}) of the supramolecular edifices follows a law inverse to the variation of the average degree of polymerization of the telomers: the greater the relative volume of the polar component, the greater the curvature of the membranes (FIG. 8). This result was confirmed by electron microscopy. Beyond p=20, all the micrographs show micellar solutions (FIG. 7b). On the other hand, below this value, they exhibit supramolecular edifices resembling oblong objects in the shape of a rice grain which do not exhibit an inner aqueous cavity in TEM after negative staining (FIG. 7a).

Using particle sizing, it appears that, for the compound of structure (C) with p=5 and R=C₁₇H₃₅, the hydrodynamic diameter of the particles obtained is 148 nm. These objects exhibit a very great stability over time, which increases proportionally to the value of p (from 2 weeks for p=5 to several months for p=25).

These ellipsoidal particles exhibit original structural characteristics and their mean hydrodynamic diameter can be readily modulated by varying p, i.e. the number of monomeric units constituting the hydrophilic polymeric part. The particles of the invention have also shown an ability to encapsulate hydrophobic active agents. This incorporation can be carried out using techniques well known to those skilled in the art. For example, the encapsulation can be carried out by dissolution of the active agent in a preformed solution of ellipsoids or of micelles, by the oil-in-water procedure or by dialysis. The therapeutic compounds which can be encapsulated are all the compounds, preferably hydrophobic compounds, which can be stably incorporated into these micellar or ellipsoid edifices. Various families of weakly hydrophilic or hydrophobic active ingredients can be encapsulated or dissolved by means of these objects, including anticancer agents, antibiotics, immunomodulators, steroids, anti-inflammatories or nucleotides. Hydrophilic compounds capable of complexing with the polar part of the nanoparticles can also be encapsulated or vectorized. The dose of active agent effectively encapsulated in these nanoparticles is determined after filtration of the nonencapsulated active agent by HPLC, by UV or fluorescence spectrometry, and also by ¹H NMR.

In addition to the compounds of formula (I), the micellar, ellipsoidal or liposome nanoparticles of the invention can also preferably contain at least one compound corresponding to formula (II) below: 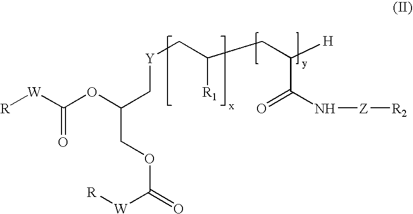
in which:
Y represents a sulfur atom or the —NH—CO—(CH₂)n-X— group in which X represents a sulfur atom S or a —CH₂— group, n is an integer ranging from 0 to 10;
W represents an —NH— or —CH₂— group;
x represents 0 or an integer ranging from 1 to 30;
y represents 0 or an integer ranging from 1 to 10;
R₁ represents a hydrophilic group chosen from the following radicals: 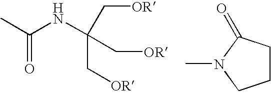
   in which R′ represents H or a hydrophilic group, such as, for example, a C₄-C₂₄ polyhydroxylated hydrocarbon-based compound; in particular R′ can be chosen from sugars, such as, for example, galactose, glucose, mannose or sialic acid, linked via its anomeric carbon;
R₂ represents a recognition group which is chosen according to the cellular target; it is preferably chosen from groups having a marked affinity for the biological target of the active ingredient transported in the nanoparticle.
R₂ may be saccharide in nature (targeting of the specific membrane lectins which are found in specific tissues, and which selectively recognize either galactose—in the case of liver, bone, certain cancerous tumors—or mannose—in the case of macrophages, the heart—or sialic acid—in the case of erythrocytes—etc.), hormonal in nature (such as steroids), synthetic in nature such as Gleevek for targeting kinases, specific antibodies, biotin, which binds to certain specific proteins, and more generally any substrate for which prior research has demonstrated recognition specificity. Among the peptides that can be used in the present invention, mention may, for example be made of the RGD sequence, known for its affinity for αVβ3 integrins.

It may be envisioned that the same molecule of formula (II) contains one or more identical recognition groups R₂ or more different recognition groups R₂, which makes it possible to direct the particles to several distinct biological targets.
The R group obeys the same rules as those defined above for the structure of the compound of formula (I).
Z is a spacer arm which connects the recognition group R₂ to the polymeric chain. Z is bound to R₂ by means of a bond which can be chosen from the functions —O—CO—, —CO—NH—, —NH—CO—NH—, —NH—CO—O—, O—CO—O—, —O—, —CH═N— or —S— or by complexation of a nickel atom (WoodleChikh et Lasical., Biochim. Biophys. Acta, 1113 (1992) 171-1999), Acta, 1567 (2002) 204-212). The latter can bind firstly to a polyhistidine tag attached to the targeting agent and, secondly, to a polyacid of NTA type attached to the polymeric chain.

The spacer arm Z can consist of a peptide chain. The latter can be attached to the oligomeric chain by means of the side chain or the main chain of the amino acid located at the end. This spacer arm comprises 1 to 5 amino acids, preferably 1 to 3 amino acids.

The amino acids constituting the spacer arm Z are chosen from natural amino acids, such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine or valine, or non-natural amino acids, such as hydroxyproline, norleucine, ornithine, citrulline or cyclohexylalanine. This spacer arm Z can consist of a tyrosine residue that makes it possible to follow the vector in vivo after labeling with ¹²⁵I or ¹³¹I.

The use of Ω-amino acids such as 3-aminopropionic acid and 4-aminobutyric acid, but also ethanolamine, 3-propanolamine or diamines of formula —NH—(CH₂)ᵣNH—, in which r represents an integer ranging from 2 to 6, can also be envisioned as group Z.

When there is binding by complexation of a nickel atom, the -Z-R₂ group consists of a group NTA of formula:

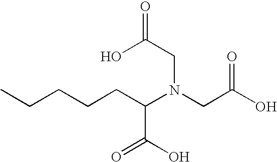

According to the first variant of the invention which concerns the liposomes formed from molecules of formula (IA), the preferred compounds of formula (II) are those of formula (IIA) below: 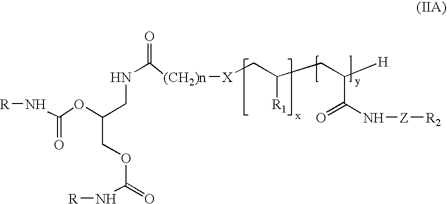
in which X, n, x, y, R, R₁ and R₂ have the same definition as above in formula (II);
preferably x and y are not zero simultaneously;
preferably X represents S;
preferably n=2.

According to the second variant of the invention which concerns the liposomes formed from molecules of formula (IB), the preferred compounds of formula (II) are those of formula (IIB) below: 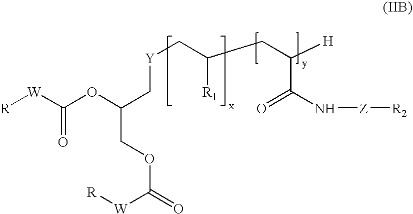
in which:
Y represents a sulfur atom or the —NH—CO—CH₂CH₂S— group;
W, x, y, Z, R, R₁ and R₂ have the same definition as in formula (II) above.

Preferably, the nanoparticles (liposomes, tubular vesicles, micelles or ellipsoidal particles) of the invention contain from 1 to 5% of one or more compounds of formula (II), which makes it possible to promote the targeting of these nanoparticles to their biological target without impairing their organization.

These lipid telomers of formula (II) have the advantage, by virtue of their oligomeric hydrophilic part, of being able to distance the grafted recognition agents from the surface of the tubular vesicles, thus promoting their recognition by the target cells or tissues. The other advantage associated with the use of these targeting lipids (II) is the possibility of multiplying the recognition units on a single compound by virtue of the telomerization technique. The factors x and y are in fact easy to control and will depend quite closely on the proportion of monomers and of telogenic agent used in the reaction.

The ligation of the recognition agents can be carried out before the telomerization of the hydrophilic head if there is compatibility with the reaction conditions. The recognition agents can also be attached to the oligomeric polar head after formation of the tubular vesicles. The telomerized hydrophilic part is then functionalized with groups capable of providing the coupling with these recognition agents. The various coupling techniques that can be used are well known to those skilled in the art and they are in particular described in: Allen et al., Biochim. Biophys. Acta, 1237 (1995) 99-108; Sapra, Prog. Lipids Res., 42 (2003) 439-462, Hansen et al., Biochim. Biophys. Acta, 1239 (1995) 133-144.

The compounds of formula (II) constitute another subject of the invention.

The compounds of formula (I) and of formula (II) described above can be grouped together under the same formula (III): 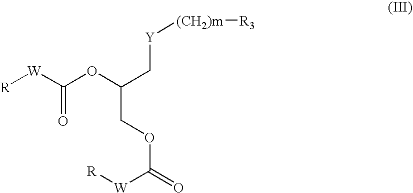
in which R, W, m and Y have the same definition as in formulae (I) and (II) above, and R₃ represents a group chosen from: 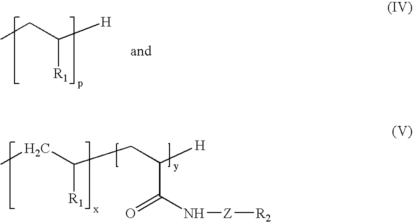
R₁ having the same definition as in formulae (I) and (II), p having the same definition as in formula (I), and x, y, Z and R₂ having the same definition as in formula (II).

In particular, the compounds of formula (IA) and of formula (IIA) can be grouped together under a common formula (IIIA): 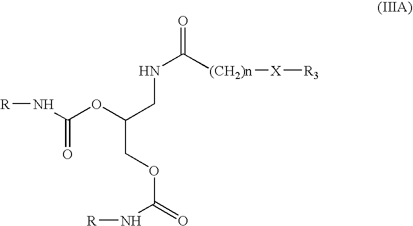
in which X, n and R have the same definition as in formulae (lA) and (IIA), and R₃ represents a group chosen from: 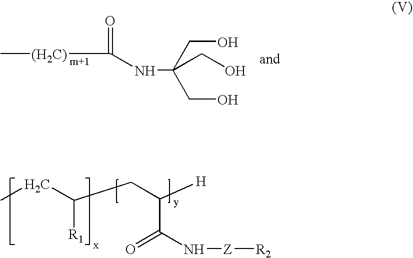
m having the same meaning as in formula (IA),
R₁, R₂, Z, x and y having the same meaning as in formula (IIA)

The compounds of formula (IB) and of formula (IIB) defined above can be grouped together under a common formula (IIIB): 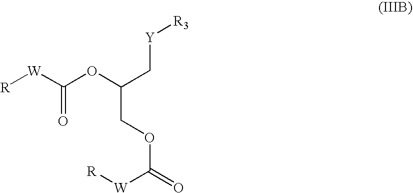
in which R, W and Y have the same definition as in the formulae (IB) and (IIB), and R₃ represents a group chosen from: 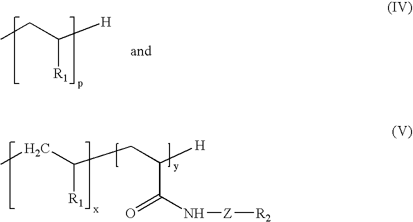
R₁ having the same definition as in formulae (IB) and (IIB), p having the same definition as in formula (IB), and x, y, Z and R₂ having the same definition as in formula (IIB).

According to a preferred variant of the invention, the liposomes or tubular vesicles of the invention formed from the compounds of formula (IA), and optionally (IIA), are stabilized by telomerization or polymerization of a monomer of acrylic type contained in their inner aqueous cavity.

In order to limit the release of the solutes encapsulated in these tubular vesicles and to increase their mechanical stability, it is possible to introduce an oligomeric or polymeric matrix into the inner aqueous compartment of the tubular vesicles. The oligomeric matrix is produced after encapsulation of the constitutive monomer(s) in the tubular vesicles and elimination of the nonencapsulated monomers by size exclusion gel separation techniques. The telomerization, which consists in forming the polymer in the presence of a chain transfer agent, makes it possible to obtain small polymers of controlled size. The low molecular mass of this polymer promotes its elimination by the kidneys. By avoiding the accumulation of polymer in the lysosome, problems of toxicity are also avoided.

The nanoparticles, liposomes or tubular vesicles comprising, in addition to the surfactants of formula (IA), at least one oligomer or telomer, as described below, constitute another subject of the invention.

The oligomer or the telomer consists of ionic or nonionic, hydrophilic monomeric construction blocks chosen from acrylic acid, methacrylic acid and methacrylamide derivatives, and also acrylate, methacrylate, acrylamide and methacrylamide derivatives, of C₁-C₆ alcohols, of C₂-C₁₂ polyols, of sugars and of amino acids.

These sugars can be:
simple sugars, such as: glucose, ribose, arabinose, xylose, lyxose, allose, altrose, mannose, galactose, fructose or talose;
disaccharides, such as maltose, sucrose or lactose.

The amino acids can be chosen from natural amino acids, such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine or valine, or non-natural amino acids, such as hydroxyproline, norleucine, ornithine, citrulline or cyclohexylalanine.

Among the commercially available monomers that can be used in the process of the invention, mention may, for example, be made of: tris(hydroxy)methylacrylamidomethane, sodium acrylate, hydroxyethyl acrylate, glucose monoacrylate, glucose-1-(N-methyl)acrylamide, glucose-2-acrylamide, maltose-1-acrylamide and sorbitol monoacrylate.

In order to modify the retention and stabilization capacities of the telomer, it is possible to use, in the production of the telomer, water-soluble crosslinking agents such as: glucose-1,2-diacrylamide, sorbitol diacrylate, sucrose diacrylate, sucrose di(ethylenediamineacrylamide), kanamycin tetracrylamide, kanamycin diacrylamide or other sugars di- or poly-functionalized with acrylates or acrylamides. As a general rule, all hydrophilic compounds capable of accepting at least two acrylate or acrylamide groups can be used. This is the case, for example, of the tris(hydroxymethyl)acrylamidomethane acrylate derivative (compound E):

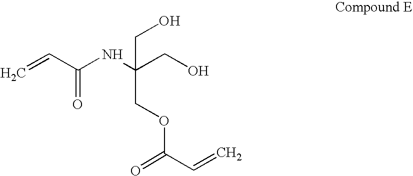

These crosslinking agents are used in proportions ranging from 1 to 5% by weight relative to the weight of the monomer(s).

The size of the telomer is controlled by using one or more hydrophilic or hydrophobic transfer agents which are inserted into the membrane or the inner aqueous cavity of the nanoparticles, and particularly of the tubular vesicles. The chain transfer agent can be hydrophilic or hydrophobic, of thiol or phosphite type. The chain transfer agents used are chosen from hydrophilic thiols, such as thiolacetic acid, mercaptopropionic acid, thioethylene glycol, cystamine or cysteine, or hydrophobic thiols, such as C₂ to C₃₀ alkanethiols for instance compound D (cholesterol derivative) which is known for its ability to integrate into phospholipid membranes. The synthesis of compound D is described in particular in M. Wathier et al., Chem. Phys. Lipids (2002), 115, 17-37.

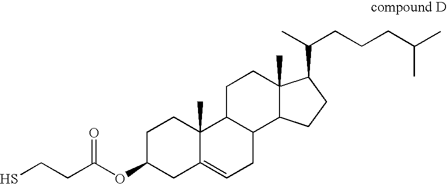

The chain transfer agent can also be chosen from the double-chained thiols previously described. The phosphites may, for their part, be hydrophilic, such as diethyl phosphite, or hydrophobic such as dioctyl phosphite, didodecyl phosphite or dihexadecyl phosphite.

In order to avoid disorganizing the membrane of the tubular vesicles, it is preferable for the hydrophobic structure of these hydrophobic thiols to be similar to that of the surfactants constituting it. The common unit of these telogenic agents advantageously consists of an aminoglycerol unit onto which are grafted fatty chains via carbamate bonds according to formula (VI): 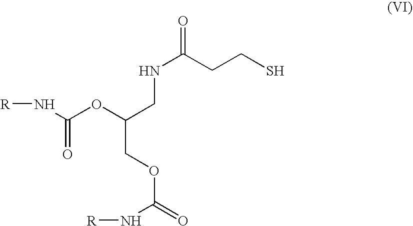
in which R has the same meaning as in formulae (I), (II) and (III) above.

In order to modulate the degree of polymerization, it is possible to vary the proportion of chain transfer agent incorporated into the membrane relative to the amount of monomers encapsulated. The chain transfer agent/lipid surfactant (I) ratio preferably ranges from 1 to 10% as weight/weight. The chain transfer agent/monomer ratio ranges from 0.1 to 10% as weight/weight.

The polymerization can be initiated in a known manner by ultraviolet irradiation or with photoinitiators, with redox initiator couples, with heat or with thermal free-radical initiators, and more generally by means of all the conventional techniques described in the literature (G. Odian, Principles of polymerization, 3. sup.rd Ed, Wiley, New York, 1991).

At the end of the polymerization, the inner aqueous cavity of the tubular vesicles contains a hydrophilic telomer that optionally has a telogenic hydrophobic part integrated into the inner membrane lamella. The physicochemical description of these stabilized tubular vesicles is given in the experimental section and their greater capacity for retention of an encapsulated solute and their increased mechanical stability are demonstrated.

The applications targeted include the transport of active ingredients, in particular of therapeutic active ingredients, the epidermal delivery of cosmetic substances, and agents for medical diagnosis; in particular, the transport of anticancer active agents, of vaccines, of genetic material, of enzymes, of hormones, of vitamins, of sugars, of proteins and peptides, of lipids, or of organic and inorganic molecules.

In the context of a vaccine-related use, the epitopes and peptides may be integrated into the inner aqueous matrix or expressed at the surface of the tubular vesicles by means of a system of covalent bonding in order to improve the immune response against the epitopes.

A subject of the present invention is therefore also any composition, in particular any therapeutic, diagnostic, vaccine or cosmetic composition, comprising at least one active ingredient in combination with a nanoparticle, liposome, tubular vesicle, ellipsoid or micelle, as described above, and in particular any composition comprising at least one active ingredient encapsulated in a liposome or tubular vesicle, ellipsoid or micelle according to the present invention.

### Experimental Section

### FIGURES

FIG. 1 illustrates the kinetics of release of carboxyfluorescein encapsulated in phosphatidylcholine liposomes (+) and tubular vesicles consisting of compound Al (●) measured by spectrofluorimetry.

FIG. 2 represents the liquid-phase infrared spectrum of a solution of compound A1 in CCl₄ of various concentrations (1×10⁻² to 2.5×10⁻⁴ M).

FIG. 3 represents the volume-based size distribution curve of the tubular vesicles, measured by electron microscopy.

FIG. 4 is a photograph obtained by phase transmission electron microscopy after negative staining with 2% uranyl acetate of tubular vesicles formed by dispersion of compound A1 (2.5 mg.ml⁻¹) in water.

FIG. 5 is a photograph obtained by phase transmission electron microscopy after freeze-fracture of a sample of tubular vesicles formed by dispersion of compound A1 (2.5 mg.ml⁻¹) in water.

FIG. 6 is a photograph obtained by phase transmission electron microscopy after negative staining with 2% uranyl acetate of a sample of tubular vesicles formed by dispersion of the compound of structure B (2.5 mg.ml⁻¹) in water.

FIG. 7 represents phase transmission electron micrographs (negative staining with 20% uranyl acetate) of aqueous dispersions of compounds of formula C with R=C₁₇H₃₅ and p=5 (a), and R=C₁₇H₃₅ and p=20 (b), and electron micrographs after freeze-fracture of the compound of formula C with R=C₁₇H₃₅ and p=8 (c).

FIG. 8 is a table grouping together the results of a physicochemical and particle size study of aqueous dispersions of the compounds of structure C with R=C₁₇H₃₅, and p variable.

FIG. 9 represents the phase transition temperatures measured by light scattering and reiteration of the values measured by spectrofluorimetry.

### EXAMPLE 1

### synthesis of the derivative A1

The synthesis of the derivative A1 is summarized in scheme 1.

Tritylmercaptopropionic Acid (1): Commercially Available Molecule.

rac-N-(2,3-Dihydroxypropyl)-3-(trityl-mercapto)propionamide (2)

2 g of 3-(tritylmercapto)propanoic acid (5.75 mmol), 0.523 g of aminopropanediol (1 eq) and 1.7 g of N-(ethoxycarbonyl)-2-ethoxy-1,2-dihydroquinoline (EEDQ) (6.9 mmol, 1.2 eq) are solubilized in 50 ml of dichloromethane. The reaction medium is refluxed for 16 hours. The reaction crude is subsequently washed with a saturated sodium bicarbonate solution and then with a normal hydrochloric acid solution saturated with sodium chloride, before being dried over sodium sulfate. After filtration over sintered glass, the product is purified by silica gel chromatography, elution being carried out with a gradient of pure ethyl acetate to ethyl acetate/methanol 9:1 (v:v). The pure product is obtained in the form of a white powder (2.12 g, yield: 87%).

The product can also be obtained pure, with an identical yield, by crystallization of the reaction crude, at ambient temperature, from an 8:2 (v:v) ethyl acetate/methanol mixture in 8 days.

¹H NMR(/CDCl₃): δ (ppm) 7.40-7.25 (15H, m, trityl aromatics); 5.86 (1H, t, NH); 3.7 (1H, m, CHOH); 3.51 (2H, m, CH₂OH); 3.31 (2H, m, CH₂NH); 3.09 (2H, m, OH); 2.50 (2H, t, CH₂S); 2.04 (2H, m, SCH₂CH₂CO). ¹³C NMR(/CHCl₃): δ (ppm) 172.9 (CH₂CONH); 144.6 (SCC phenyl); 129.6 (Cₚₐᵣₐ phenyl); 128.0 (Cortho phenyl); 126.8 (Cₘₑₜₐ phenyl); 70.9 (CHOH); 66.9 (SCPh₃); 63.6 (CH₂OH); 42.1 (NHCH₂CH); 35.3 (SCH₂CH₂CO); 27.6 (CH₂S).

Rac-2,3-di[N-(heptadecyl)carbamoyloxypropyl]-3-(tritylmercapto)propionamide (3)

2 g of rac-N-(2,3-dihydroxypropyl)-3-(tritylmercapto) propionamide (4.75 mmol) and 2.8 g of 1-isocyanato-heptadecane (9.97 mmol, 2.1 eq) are solubilized in 50 ml of freshly distilled toluene, at ambient temperature and under sparging with argon. The reaction medium is brought to reflux and a spatula tip of 1,4-diazabicyclo[2,2,2]octane (DABCO) (cat.) is added to the mixture. After 6 hours, the reaction crude is evaporated to dryness and taken up in a minimum amount of ether, from which the product crystallizes at ambient temperature. After filtration, the product is obtained pure in the form of a white powder (4.04 g, yield: 86%).

¹H NMR(CDCl₃): δ (ppm) 7.47-7.23 (15H, m, trityl aromatics); 6.02 (1H, t, NH); 4.95-4.74 (3H, m, CHO, CH₂O); 4.18 (2H, m, NH); 3.44 (2H, t, CH₂NH); 3.12 (4H, q, CH₂NH); 2.51 (2H, t, CH₂S); 2.06 (2H, t, SCH₂CH₂CO); 1.49 (4H, m, NHCH₂CH₂); 1.28 (54H, m, CH₂ chains); 0.91 (6H, t, CH₃). ¹³C NMR(CDCl₃): δ (ppm) 171.2 (CH₂CONH); 156.1-155.9 (OCONH); 144.7 (SCC phenyl); 129.6 (Cₚₐᵣₐ phenyl); 127.9 (Cₒᵣₜₕₒ phenyl); 126.7 (Cₘₑₜₐ phenyl); 71.2 (CHO); 66.8 (SCPh₃); 63.4 (CH₂O); 41.2 (NCH₂CH₂); 40.0 (NHCH₂CH); 35.5 (SCH₂CH₂CO); 31.9 (NCH₂CH₂); 29.9-29.3 (chains); 27.6 (CH₂S); 22.7 (CH₂CH₃); 14.1 (CH₃).

Rac-2,3-di[N-(heptadecyl)carbamoyloxypropyl]-3-mercaptopropionamide (4)

2 g of compound 3 (2.03 mmol) and 0.236 g of triethylsilane (2.03 mmol, 1 eq) are solubilized in a minimum amount of dichloromethane and cooled to 0° C. with an ice bath. A 10% solution of trifluoroacetic acid in dichloromethane is added, under cold conditions, dropwise, via a dropping funnel. As soon as the addition is complete, the medium is returned to ambient temperature and left for 3 hours with stirring. After evaporation to dryness, taking up in dichloromethane and washing with sodium chloride-saturated distilled water and then with a saturated sodium bicarbonate solution, the reaction crude is evaporated to dryness and taken up in ethyl acetate, from where it crystallizes under cold conditions. The pure product is recovered in the form of a white powder (1.3 g, yield: 86%).

¹H NMR(CDCl₃): δ (ppm) 6.46 (1H, t, NH); 4.97-4.95 (3H, m, CHO, CH₂O); 4.21 (2H, m, NH); 3.50 (2H, t, CH₂NH); 3.16 (4H, q, CH₂NH); 2.81 (2H, q, CH₂S); 2.51 (2H, t, SCH₂CH₂CO); 1.64 (1H, t, SH); 1.50 (4H, m, NHCH₂CH₂); 1.27 (54H, m, CH₂ chains); 0.89 (6H, t, CH₃). ¹³C NMR (CDCl₃): δ (ppm) 170.9 (CH₂CONH); 156.1-156.0 (OCONH); 71.3 (CHO); 63.5 (CH₂O); 41.2 (NCH₂CH₂); 40.4 (NHCH₂CH); 35.5 (SCH₂CH₂CO); 31.9 (NCH₂CH₂); 29.9-29.4 (chains); 22.7 (CH₂CH₃); 20.4 (CH₂S); 14.1 (CH₃).

Acetylated compound A1

0.5 g of compound 4 (0.67 mmol) and 1.01 g of triacetylated THAM (3.37 mmol, 5 eq) are solubilized in 30 ml of freshly distilled triethylamine. The mixture is brought to 50° C. under sparging with argon for 2 hours and then returned to ambient temperature and evaporated to dryness. The reaction crude is taken up in ethyl acetate so as to be washed with a normal aqueous solution of hydrochloric acid and then with a saturated aqueous sodium bicarbonate solution. The product is subsequently purified by silica gel chromatography on a column eluted with pure ethyl acetate. After evaporation and drying, the product is obtained pure in the form of a white powder (0.33 g, yield: 46%).

¹H NMR(/CDCl₃): δ (ppm) 6.48 (1H, t, CONHCH₂); 6.37 (1H, s, CONHC); 5.05-4.96 (3H, m, CHO, CH₂O); 4.45 (6H, s, CH₂O); 4.22 (2H, m, NH); 3.48 (2H, t, CH₂NH); 3.17 (4H, q, CH₂NH); 2.81 (2H, q, CH₂S); 2.49 (4H, t, SCH₂CH₂CO); 2.10 (9H, s, CH₃); 1.44 (4H, m, NHCH₂CH₂); 1.27 (54H, m, CH₂ chains); 0.90 (6H, t, CH₃). ¹³C NMR(/CDCl₃): δ (ppm) 171.7-171.5 (CH₂CONH); 170.6 (OCO); 156.2-156.0 (OCONH); 71.2 (CHO); 63.6 (CH₂O); 62.5 (CH₂O); 41.2 (NCH₂CH₂); 40.2 (NHCH₂CH); 37.1-36.6 (SCH₂CH₂CO); 31.9 (NCH₂CH₂); 29.8-29.3 (chains); 26.8 (CH₃); 22.6 (CH₂CH₃); 20.8 (CH₂S); 14.1 (CH₃). 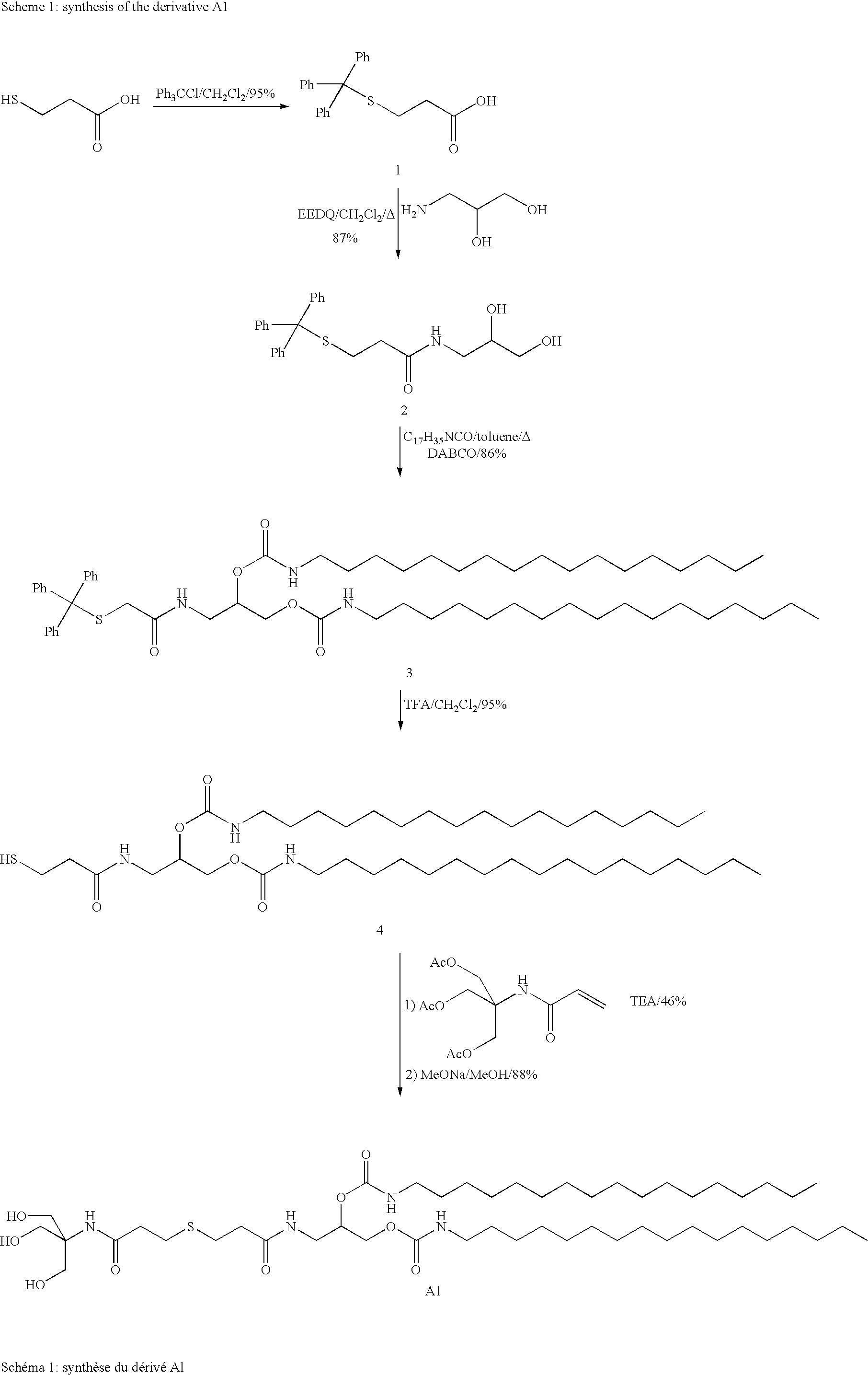
Compound A1

0.3 g of acetylated derivative (0.28 mmol) are solubilized in a minimum amount of methanol and then a spatula tip of sodium methoxide (cat.) is added. The mixture is left at ambient temperature for 30 minutes, with stirring, while maintaining the pH between 8 and 9 by the addition of sodium methoxide if necessary. The reaction medium is subsequently neutralized by the addition of a few drops of a normal aqueous solution of hydrochloric acid. After evaporation to dryness, the reaction crude is purified by silica gel chromatography, elution being carried out with a 95:5 (v:v) ethyl acetate/methanol mixture. The product is obtained pure in the form of a white powder (0.233 g, yield: 88%). Mp: 129° C.

¹H NMR(CDC1₃): δ (ppm) 7.00 (1H, s, CONHC); 6.72 (1H, t, CONHCH₂); 5.00 (3H, m, CHO, CH₂O); 4.47 (3H, m, OH×3); 4.44 (6H, s, CH₂O×3); 4.19 (2H, m, NH×2); 3.48 (2H, t, CH₂NH); 3.16 (4H, q, CH₂NH×2); 2.85 (2H, q, CH₂S); 2.65-2.50 (4H, t, SCH₂CH₂CO×2); 1.44 (4H, m, NHCH₂CH₂×2); 1.28 (54H, m, CH₂ chains); 0.91 (6H, t, CH₃×2). ¹³C NMR(CDCl₃): δ (ppm) 171.1 (CH₂CONH); 156.2-156.0 (OCONH); 71.2 (CHO); 63.6 (CH₂O); 61.0 (CH₂OH); 41.2 (NCH₂CH₂); 40.2 (NHCH₂CH); 37.1-36.6 (SCH₂CH₂CO); 31.9 (NCH₂CH₂); 29.9-29.3 (chains); 22.6 (CH₂CH₃); 20.41 (CH₂S); 14.1 (CH₃).

### EXAMPLE 2

### Synthesis of the Crosslinking Agent E: 2-acryloylamino-3-hydroxy-2-(hydroxymethyl)propyl ester of acrylic acid

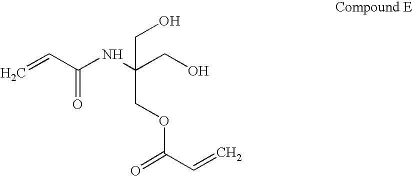 5-Acryloylamino-2,2-dimethyl[1,3]dioxan-5-ylmethyl ester of acrylic acid

1 g (4.65 mmol) of THAM isopropylidene is solubilized in a minimum amount of dichloromethane. The pH is adjusted and maintained at 9 by the addition of a few drops of triethylamine and then a solution containing 0.378 ml of acryloyl chloride (4.65 mmol, 1 eq) in 4 ml of dichloromethane is added dropwise. The reaction is monitored by TLC (7:3 ethyl acetate/cyclohexane) with disappearance of the THAM isopropylidene (Rf: 0.3). The medium is subsequently neutralized by the addition of formic acid, evaporated to dryness and purified by silica gel chromatography using a 7:3 to 5:5 ethyl acetate/cyclohexane elution gradient. The pure product is obtained in the form of a yellow oil (1.1 g, yield: 87%).

¹H NMR(CDCl₃): δ (ppm) 6.50 (m, 1H, CHCONH), 6.43 (d, 1H, CHC_{2b} acrylic ester), 6.30 (s, 1H, NH); 6.22 (d, 1H, CH_{2b} acrylamide), 6.14 (d, 1H, CHCOO), 5.93 (d, 1H, CH₂ₐ acrylic ester), 5.65 (d, 1H, CH₂ₐ acrylamide), 4.62 (s, 2H, CH₂OCO), 4.42 (d, 2H, CH₂O), 3.80 (d, 2H, CH₂O), 1.49 (d, 6H, CH₃×2)

2-Acryloylamino-3-hydroxy-2-(hydroxymethyl)propyl ester of acrylic acid

1.1 g (4.08 mmol) of 5-acryloylamino-2,2-dimethyl[1,3]dioxan-5-ylmethyl ester of acrylic acid and 5.45 g of Montmorillonite K10 are mixed in dichloromethane and left at ambient temperature for 4 days, with stirring. This mixture is subsequently filtered over celite and washed with methanol. The celite cake is taken up several times in methanol with vigorous stirring before again being filtered. After evaporation to dryness, the product is obtained pure in the form of a yellow oil (0.766 g, yield: 82%).

¹H NMR(CDCl₃): δ (ppm) 6.73 (s, 1H, NH), 6.42 (d, 1H, CH_{2b} acrylic ester), 6.21 (d, 1H, CH_{2b} acrylamide), 6.14 (d, 1H, CHCOO), 5.88 (d, 1H, CH₂ₐ acrylic ester), 5.69 (d, 1H, CH₂ₐ acrylamide), 4.70 (m, 2H, OH×2), 4.39 (d, 2H, CH₂O), 3.70 (dd, 4H, CH₂OH).

### EXAMPLE 3

### Synthesis of a Targeting Lipid Telomer G

This synthesis is illustrated by scheme 2. 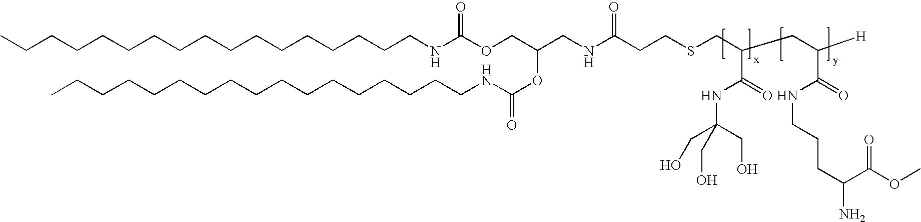
Methyl ester of 5-acryloylamino-2-tert-butyloxycarbonylaminopentanoic acid (5)

4.00 g of the acetate salt of L-(Boc)LysOMe (12.50 mmol-1 eq.) are dissolved in 30 ml of anhydrous dichloromethane. The pH of the solution is brought to 9 by the addition of DIEA and then cooled to 0° C. 1.87 ml of acryloyl chloride (23 mmol-1.85 eq.) are added dropwise to the reaction mixture while maintaining the pH of the solution basic by the addition of DIEA. After stirring for 24 hours, the medium is washed with water and the organic phase is then dried over Na₂SO₄. The solvents are evaporated off under reduced pressure and purification by flash chromatography on silica gel (eluent: 8:2 ethyl acetate/cyclohexane) makes it possible to obtain compound 5 (3.67 g, yield: 76%) in the form of a translucent colorless oil. [α]_{D}=+7.5(c, l, CHCl₃).

¹H NMR (250 MHz, DMSO-d6): δ 8.08 (1H, t, J=5.6 Hz, NH—CO—O), 7.22 (1H, d, J=7.7 Hz, NH—CO), 6.20 (1H, dd, J_{cis}=9.7 Hz and Jₜᵣₐₙₛ=17.1 Hz, H_{c}), 6.05 (1H, dd, J_{gem}=2.7 Hz and Jₜᵣₐₙₛ=17.1 Hz, H_{b}), 5.56 (1H, dd, J_{gem}=2.7 Hz and J_{cis}=9.7 Hz, Hₐ), 3.92 (1H, m, CH), 3.62 (3H, s, CH₃—O), 3.10 (2H, q, J=6.3 Hz, CH₂—NH), 1.60 (6H, m, CH₂ of lysine), 1.38 (9H, s, CH₃ of Boc). ¹³C NMR (62.86 MHz, CDCl₃): δ 169.3, 161.3 (CO), 136.8 (C^{IV} arom), 130.7 (CH arom.), 128.6 (C^{IV} arom), 128.6 (CH arom.)m, 125.5 (CH═N(O)), 72.2 (C^{IV}), 28.4 (CH₂—CO), 25.7 (CH₃ of tert-butyl). 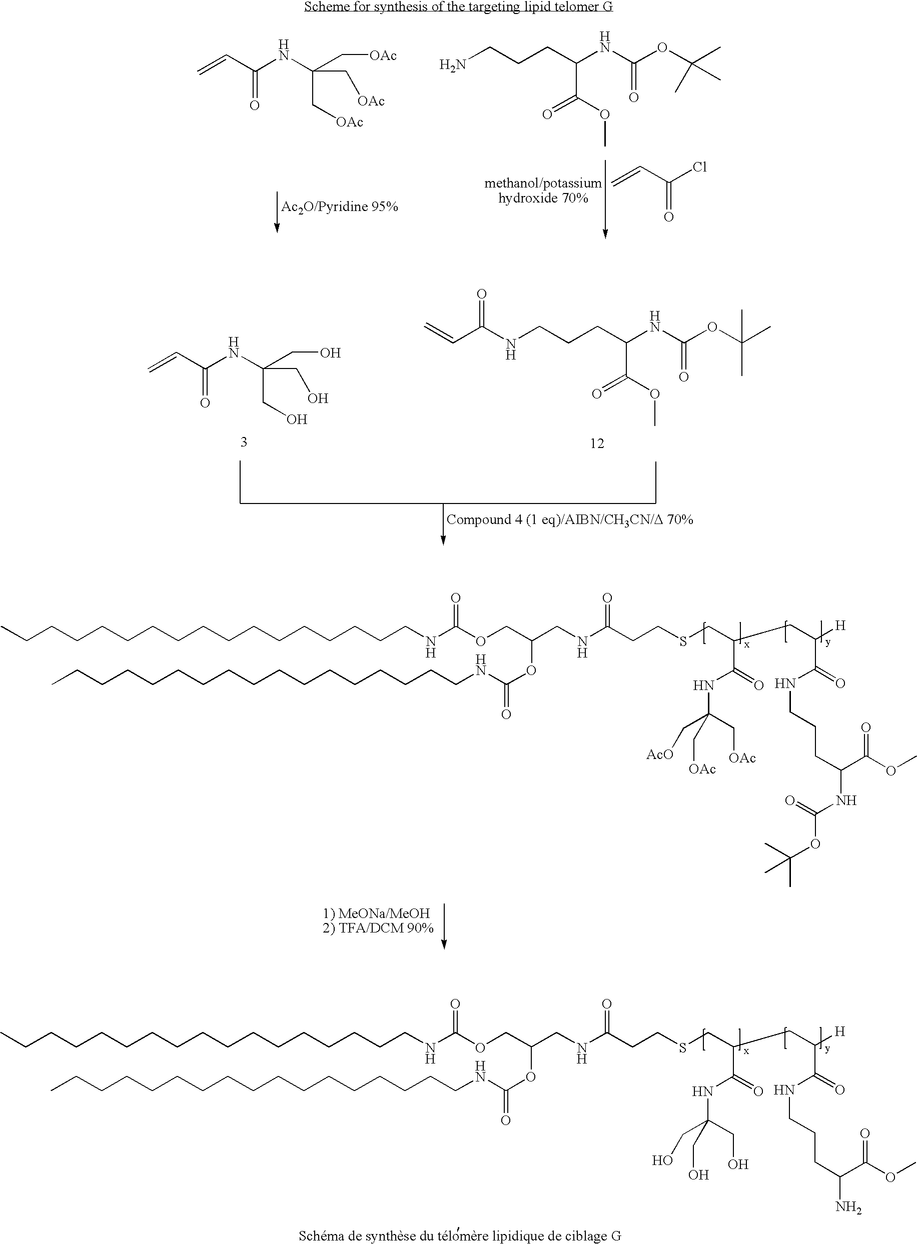
Synthesis of the Telomerized Lipid G

0.767 g of tris(acetoxymethyl)acrylamidomethane monomer 6 (2.55 mmol, 12 eq) and 0.2 g of monomer 5 (0.63 mmol, 3 eq) are dissolved in 20 ml of freshly distilled 10 acetonitrile, in a 100 ml two-necked round-bottomed flask surmounted by a condenser. The tris-(acetoxymethyl)acrylamidomethane 6 was prepared in accordance with the teaching of the document A. Polidori et al., New J. Chem. 1994, 18, 839-848. The reaction medium is degassed under argon and brought to reflux. 7 mg of AIBN (4.29×10⁻² mmol, 0.2 eq) and 0.157 g of thiol 4 (0.21 mmol, 1 eq) dissolved in 5 ml of freshly distilled and degassed acetonitrile are added. The reaction is refluxed for 4 h until the monomers have been completely used up (detected by TLC). The reaction medium is concentrated under reduced pressure and the reaction crude is filtered over a sephadex column (1:1 MeOH/CH₂Cl₂). The product is subsequently dissolved in 100 ml of methanol in the presence of a catalytic amount of sodium methoxide. After stirring for 5 h, the reaction medium is neutralized by the addition of IRC 50 acidic resin. The resin is eliminated by filtration and the solvent is eliminated under reduced pressure. The product is subsequently reacted, under cold conditions, in an acidic mixture of TFA/CH₂Cl₂ (20%) for 3 h.

The reaction medium is concentrated under reduced pressure. The oil obtained is taken up several times in ether until the telomer precipitates in the form of a white powder. The product is dissolved in water and lyophilized until the compound G is obtained in the form of a white powder.

The average degree of polymerization (DPn) and the ratio of the concentrations of each monomer in the macromolecule (x and y) were determined by ¹H NMR comparing the integrations of the signals from the methyls of the two alkyl chains at 1.1 ppm to that of the signals from Tris(CH₂OH) at 4.3 ppm and from lysine (CH₂NH) at 3.37 ppm. We were able to determine the following values for x and y:

x:30 and y:10.

### EXAMPLE 4

### Synthesis of a Targeting Lipid F

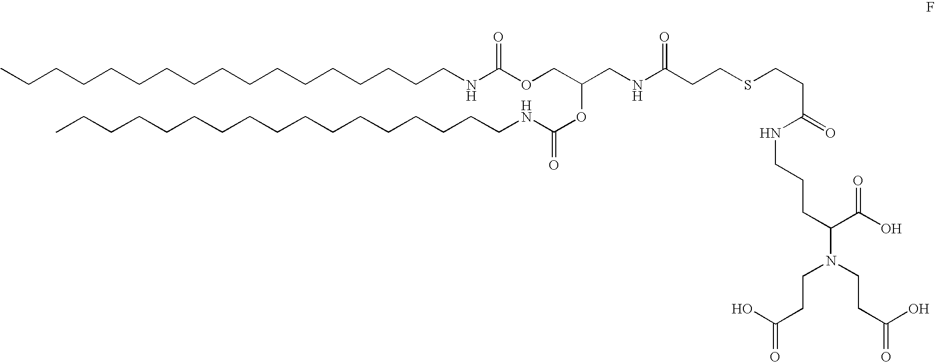 5-Acryloylamino-2-tert-butoxycarbonylaminopentanoic acid 7

2 g of Boc lysine (8.13 mmol) are dissolved in 10 ml of a 1:1 acetonitrile-2N sodium hydroxide mixture. The reaction medium is cooled to 10° C. Acryloyl chloride (1.1 g, 12.19 mmol) dissolved in 10 ml of acetonitrile is added dropwise. The pH is maintained at 8 by the addition of 2N sodium hydroxide. At the end of the addition, the reaction medium is stirred at ambient temperature for 2 h and then acidified with a 2N HCl solution, and extracted with ethyl acetate (3×20 ml). The organic phase is dried, and then concentrated under reduced pressure. The final product is obtained pure in the form of a white powder by recrystallization from ethanol (1.9 g, 78%).

¹H NMR (250 MHz, DMSO-d6): δ 8.02 (1H, t, NH—CO—O), 7.18 (1H, d, J=7.4 Hz, NH—CO), 6.25 (lH, dd, J_{cis}=9.4 Hz and Jₜᵣₐₙₛ=17.1 Hz, H,), 6.05 (1H, dd, J_{gem}=2.7 Hz and Jₜᵣₐₙₛ=17.1 Hz, H_{b}), 5.6 (1H, dd, J_{gem}=2.7 Hz and J_{cis}=9.4 Hz, Hₐ), 3.92 (1H, m, CH), 3.10 (2H, q, J=6.3 Hz, CH₂—NH), 1.60 (6H, m, CH₂ of lysine), 1.38 (9H, s, CH₃ of Boc). ¹³C NMR (62.86 MHz, DMSO-d6): δ 169.3, 161.3 (CO), 155.8 (CO urethane), 130.7 (CH₂═), 128.6 (—CH═), 80.5 (C tBu), 53.7 (CH Lys), 40.3 (CεLys), 33.2 (CβLys), 29.8 (CδLys), 28.4 (CH₂—CO), 25.7 (CH₃ of tert-butyl).

6-Acryloylamino-2-[bis(2-carboxyethyl)amino]hexanoic acid 8

1.9 g of compound 7 (6.33 mmol) are dissolved in 20 ml of a 1:1 trifluoroacetic acid-dichloromethane mixture, in a 50 ml round-bottomed flask. After stirring for 2 h, the solvent is evaporated out under reduced pressure and the oil obtained is taken up several times in chloroform and evaporated until a powder is obtained. 1.94 g of bromoacetic acid (12.66 mmol) are dissolved in 7 ml of 2N sodium hydroxide, in a 25 ml round-bottomed flask. The solution is cooled to 0° C. in an ice bath and the powder obtained after deprotection, dissolved in 11 ml of an aqueous 2N sodium hydroxide solution is added dropwise. After reaction for 2 h at ambient temperature, an aqueous 2N HCl solution is added until an acidic pH is obtained. The product precipitates. The precipitate is filtered off and filter-dried and then dried under reduced pressure. The product 8 is obtained pure in the form of a white powder after recrystallization from ethanol (1.6 g, 57%).

1H NMR (250 MHz, DMSO-d6): δ 8.02 (1H, t, NH—CO—O), 7.18 (1H, d, J=7.4 Hz, NH—CO), 6.25 (1H, dd, J_{cis}=9.4 Hz and Jₜᵣₐₙₛ=17.1 Hz, H_{c}), 6.05 (1H, dd, J_{gem}=2.7 Hz and Jₜᵣₐₙₛ=17.1 Hz, H_{b}), 5.6 (1H, dd, J_{gem}=2.7 Hz and J_{cis}=9.4 Hz, Hₐ), 3.5(4H, s, N—CH₂—COOH), 3.3 (1H, t, J=7.1 Hz, CH₂—CH—N), 2.9 (2H, m, CH₂NH), 1-1.5 (6H, m, CH₂CH₂CH₂). ¹³C NMR (62.86 MHz, DMSO-d6): δ 174.8, 173.6 (COOH), 168.4 (CONH), 130.4 (CH₂═), 128.2 (—CH═), 64.8 (CH Lys), 53.8 (N—CH₂—COOH), 40.3 (CεLys), 30.5 (CβLys), 29.8 (CδLys).

Synthesis of the Lipid F

2.56 g of compound 4 (3.6 mmol) and 1.6 g of compound 8 (3.6 mmol) are solubilized in 30 ml of freshly distilled triethylamine. The mixture is brought to 50° C. under sparging with argon, for 2 hours, and then returned to ambient temperature and evaporated to dryness under reduced pressure. The product is obtained pure in the form of a white powder after successive recrystallization from ethyl acetate (2.29 g, yield: 55%).

¹H NMR (250 MHz, DMSO-d6): δ (ppm) 7.12 (1H, t, NH—CO—O), 6.48 (1H, t, CONHCH₂); 6.37 (1H, s, CONHC); 5.05-4.96 (3H, m, CHO, CH₂O); 4.45 (6H, s, CH₂O); 4.22 (2H, m, NH); 3.48 (6H, s+t, N—CH₂—COOH, CH₂NH); 3.25 (1H, t, J=7.1 Hz, CH₂—CH—N), 3.17 (2H, m, CH₂NH); 2.81 (4H, t, CH₂S); 1.44 (4H, m, NHCH₂CH₂); 1.27 (60H, m, CH₂Lys, CH₂ chains); 0.90 (6H, t, CH₃). ¹³C NMR (250 MHz, DMSO-d6): δ (ppm) 171.7-171.5 (COOH, CH₂CONH); 156.2-156.0 (OCONH); 71.2 (CHO); 64.5 (CH Lys), 63.6 (CH₂O); 62.5 (CH₂O); 53.2 (N—CH₂—COOH), 41.2 (NCH₂CH₂); 40.2, 40.1 (CεLys, NHCH₂CH); 37.1-36.6 (SCH₂CH₂CO); 31.9 (CβLys); 29.8-29.3 (chains and CβLys), 26.8 (CH₃); 22.6 (CH₂CH₃); 20.8 (CH₂S); 14.1 (CH₃).

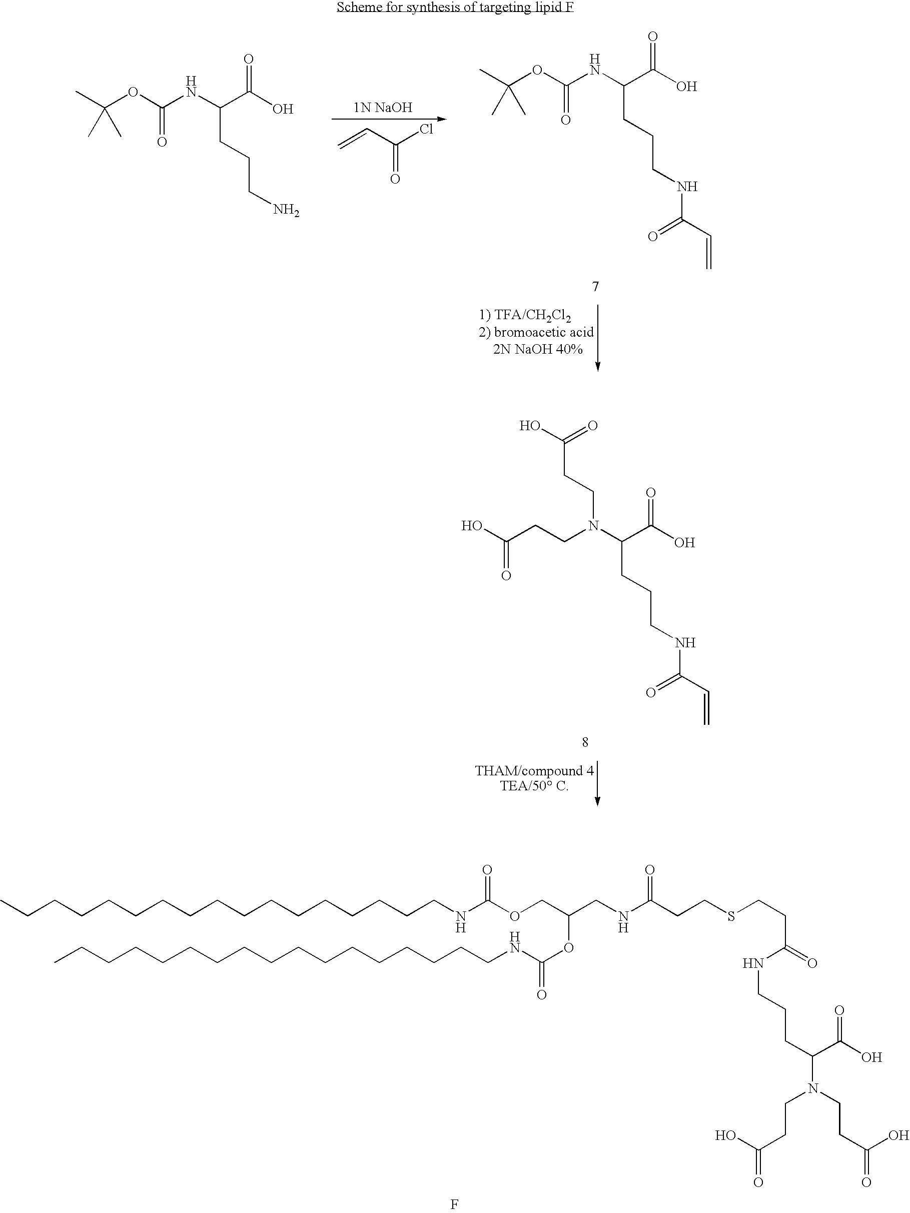

### EXAMPLE 5

### Preparation of the Tubular Vesicles from the Derivative A1

Material Used

Measurement of the Size of the Tubular Vesicles.

The size distribution of the particles was measured by photon correlation spectroscopy of light of samples diluted in water using a Malvern HPPS/NIBS device equipped with a 3 mW He/Ne laser and a 288-channel correlator equipped with an avalanche of photodiodes. The mathematical processing of the autocorrelation curve uses the Contin method.

Lipid Dispersion

The lipids A are dispersed in water according to the film method in a thermostated Bransonic sonication bath type 2510 E (100 W, 42 Hz) above their phase transition temperature for half an hour.

Formation of the Tubular Vesicle Dispersions

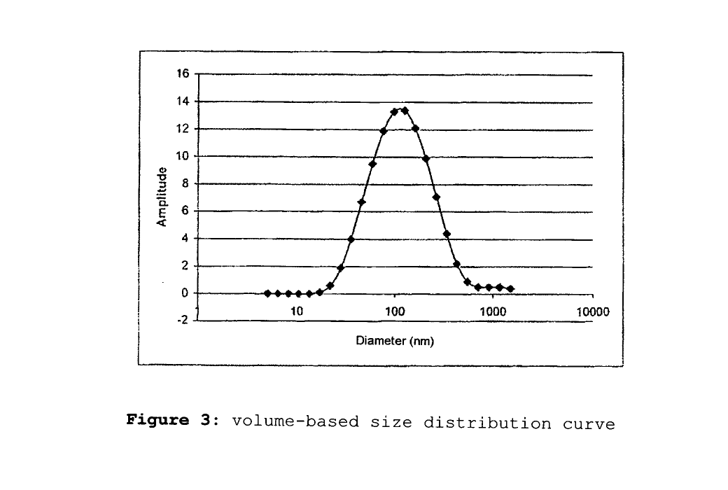

The tubular vesicles are prepared, firstly, by solubilizing the lipid A1 in chloroform. The solution is slowly concentrated under reduced pressure in a 20 ml heart-shaped round-bottomed flask using a rotary evaporator. The film obtained is subsequently dried under reduced pressure using a vane pump. The lipid film is rehydrated with distilled water at 65° C. (10° C. above the phase transition temperature of the lipid) at a concentration of 2.5 mg.ml⁻¹. The mixture is homogenized on a vortex for 5 minutes and then subjected to ultrasound for 30 minutes at 70° C. The translucent bluish solution obtained is filtered through a 0.45 pm filter and then analyzed on a photon correlation spectrometer and by transmission electron microscopy (FIGS. 3, 4 and 5).

With R=C₁₇H₃₅ (derivative A1). The phase transition temperature was measured via detection of the polarization of a fluorescent probe, DPH, by spectrofluorimetry: Tm=54° C.

We obtain tubular vesicles, the mean hydrodynamic diameter of which, measured by light diffraction, is 132 nm (IP: 0.35).

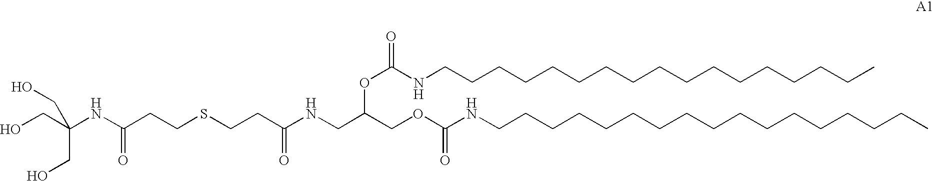

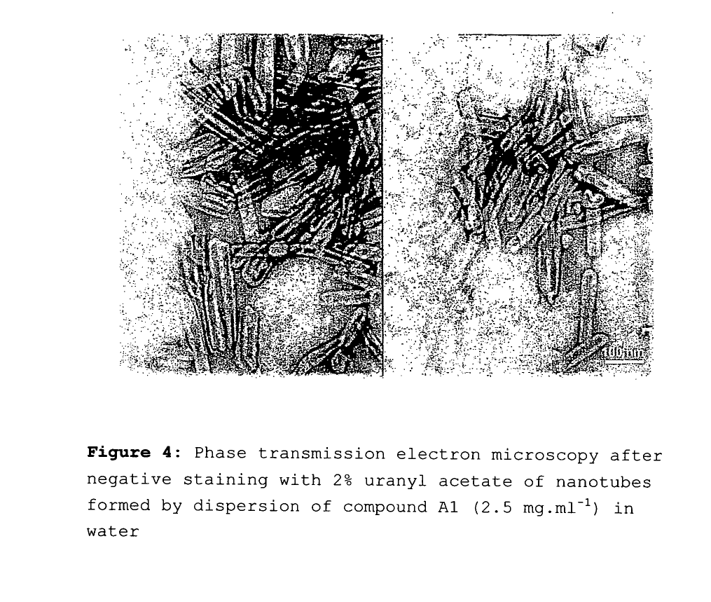

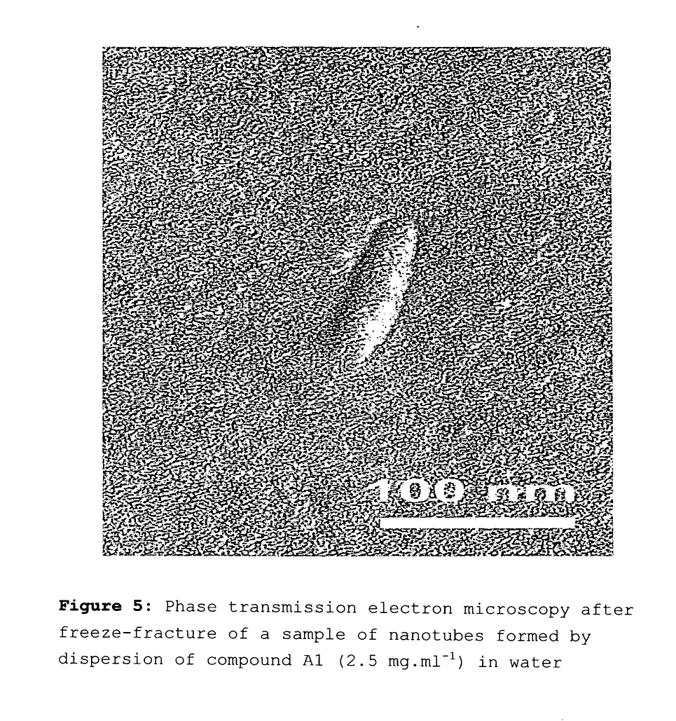

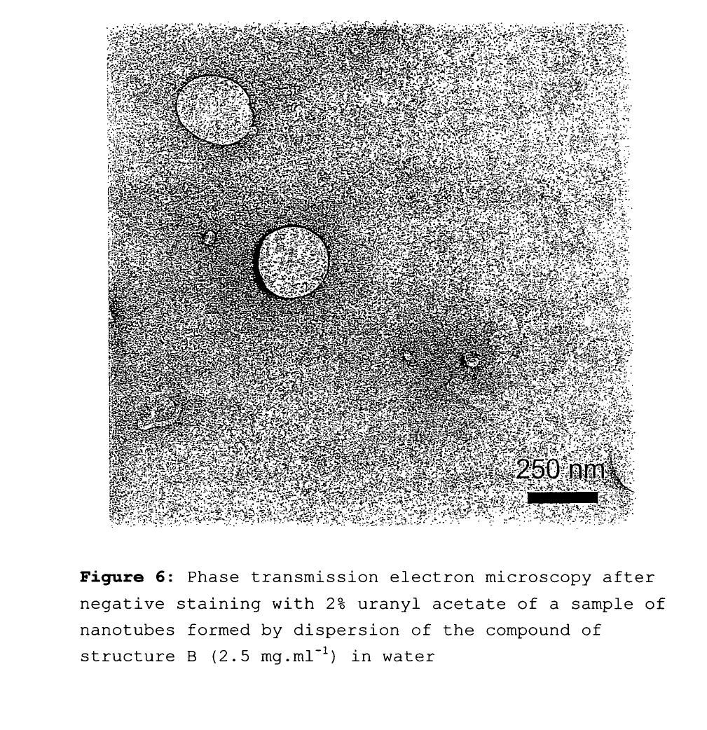

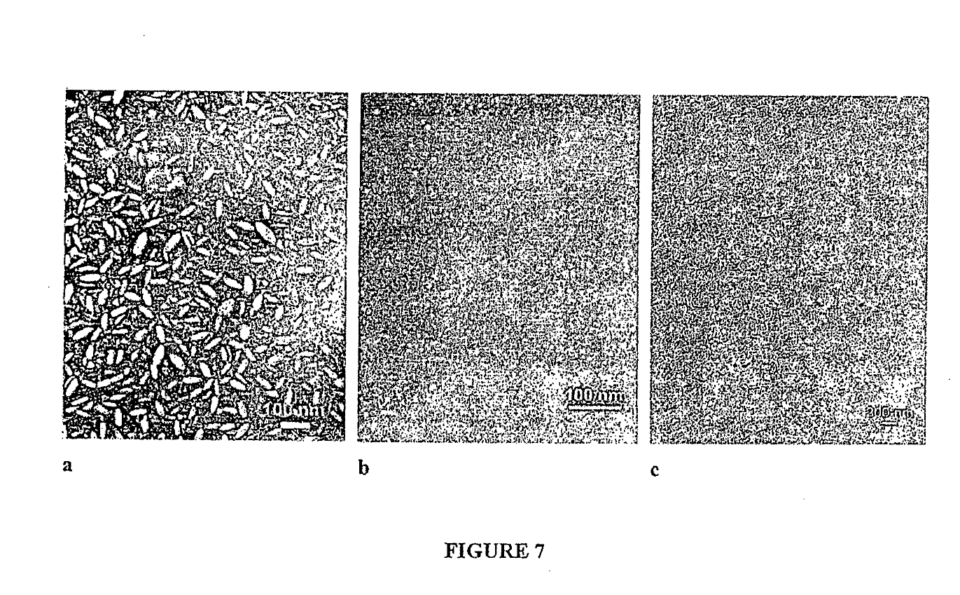

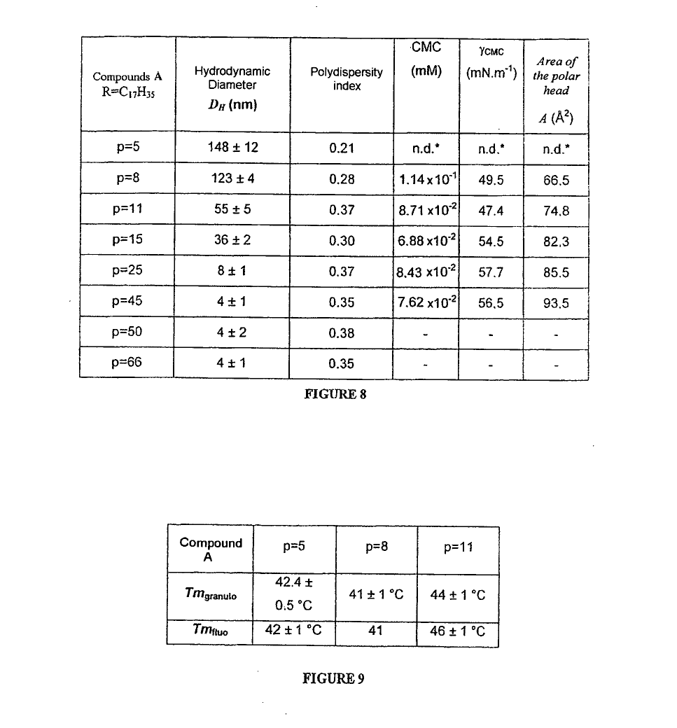

The measurements of shape and size established by observation of the electron micrographs (FIGS. 4 and 5) made it possible to demonstrate the formation of tubular vesicles closed up at their ends, the average cross section of which is 38 nm and the average length of which is 247 nm. The freeze-fracture analyses confirmed the formation of these tubular vesicles and their morphological characteristics, i.e. the presence of an inner aqueous cavity isolated from the outside medium.

These tubular vesicles have a high stability since no change in particle size is observed after one year of storage, whereas, under the same conditions, liposomes formed from egg yolk phosphatidylcholine change after only 5 days.

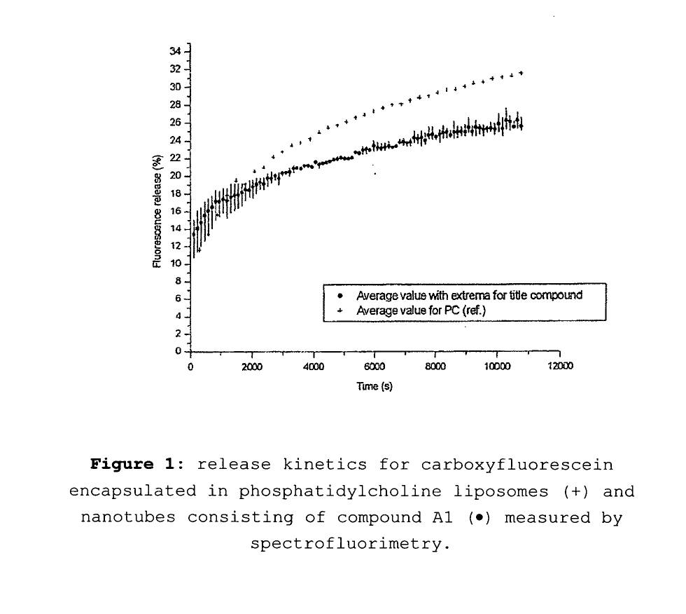

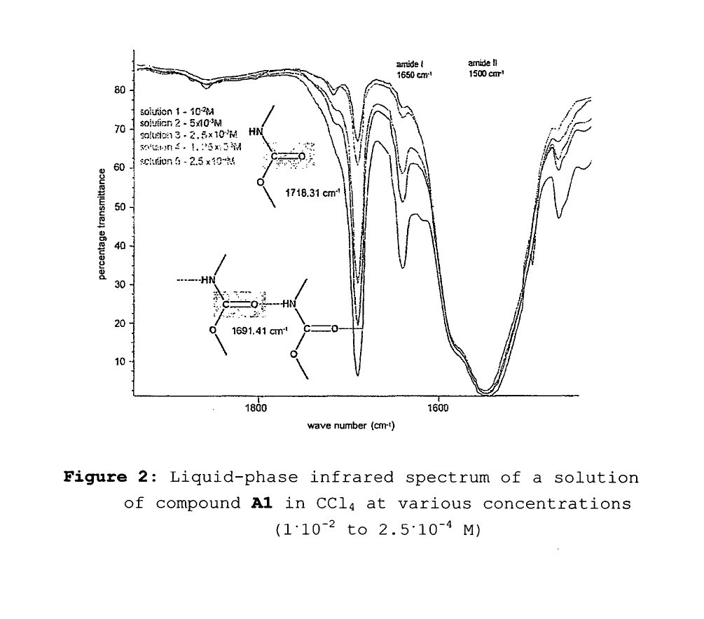

The demonstration of the aqueous inner cavity was proved indirectly by spectrofluorimetric measurements of the encapsulation and release kinetics for a hydrophilic fluorescent probe, carboxyfluorescein (FIG. 1). The measurements clearly show slower release kinetics for the fluorescent probe compared with a conventional encapsulation in a mixture of egg yolk phosphatidylcholine.

### EXAMPLE 6

### Encapsulation of Carboxyfluorescein in Tubular Vesicles Produced from the Derivative A1

The encapsulation capacity of the various compounds is determined by spectrofluorimetry using a fluorescent probe: 5 (6)-carboxyfluorescein. The release of this fluorescent label from the vesicles prepared according to standardized methods is measured.

This study requires the preparation of a Tris buffer (15 mM and 150 mM NaCl) at pH 7.4, and also of a 120 mM carboxyfluorescein solution, also at pH 7.4.

The compounds studied are weighed out (2.5 mg/ml) and are dissolved in a minimum amount of methanol. The solvent is evaporated off under reduced pressure in a rotary evaporator and the film thus obtained is dried with a stream of nitrogen.

The 120 mM solution of carboxyfluorescein in the Tris buffer is added so as to obtain a dispersion of lipids having a concentration equal to 2.5 mg/ml. The suspension obtained is vortexed for 1 minute and then subjected to ultrasound in a sonication bath for 1 hour at 70° C.

The nonencapsulated fluorescent probe is eliminated by passage over a Sephadex G25 column pre-equilibrated with the Tris buffer. The vesicle fraction harvested is immediately studied by spectrofluorimetry.

The fluorescence measurements were carried out using a Jobin-Yvon spectrofluorimeter (spectrofluoromax 2), equipped with a 150 W xenon lamp. All the measurements were carried out in a quartz cuvette thermostated at 25° C. The samples were analyzed at an excitation wavelength of 480 nm, and an emission wavelength of 530 nm for 4 hours.

The bandwidths were fixed at 0.5 nm both for excitation and for emission.

For each measurement, the initial fluorescence intensity (F₀) is determined 30 seconds after the column filtration. The release is studied over a period of 4 h and the fluorescence intensity (Fₜ) is measured at regular intervals. The maximum fluorescence intensity (Fₘₐₓ), which corresponds to 100% release, is obtained after tubular vesicle lysis obtained by the addition of Triton X100 (10% v/v).

The percentage release is obtained by means of the following equation (FIG. 1):

% R=(Fₜ-F₀)/Fₘₐₓ

### EXAMPLE 7

### Polymerization of a Monomer Encapsulated in Tubular Vesicles Obtained from the Lipid A1

The composition of the solutions used is summarized in Table 1.

TABLE 1

| | Designation | Solvent | Concentration |
|---|---|---|---|
| Monomer | THAM | Distilled water | 0.1 M |
| | Acrylamide | Distilled water | 0.1 M |
| Initiator | tBuOOH/Na₂S₂O₅ | Dichloromethane/ | 2.5 × 10⁻⁴ M/ |
| | | buffer | 2.5 × 10⁻⁴ M |
| | Na₂S₂O₅ | Buffer | |
| Buffer | NaCl | Distilled water | 0.1 M |

Preparation of the Film

20 mg of lipids A1 are solubilized in 2 ml of a solution of cumene hydroperoxide in dichloromethane that has been freshly distilled and degassed by sparging with argon (2.5×10⁻⁴ M), in a heart-shaped round-bottomed flask.

The solution is evaporated to dryness in a rotary evaporator (the bath temperature not exceeding 40° C.) and the film is then dried with a vane pump (1 hour) and placed under an inert atmosphere until use. In the case of an initiation with sodium dithionite, the film is prepared in dichloromethane that has been freshly distilled and degassed by sparging with argon.

Dispersion

2 ml of solution of monomer in distilled water (0.1 M), deoxygenated beforehand by sparging with argon, are added. The mixture is stirred for 1 minute and then placed in an ultrasound bath for 60 minutes at 70° C.

Separation-polymerization

The preparation is loaded onto a Sephadex G50 column (2 cm in diameter for a height of 10 cm of gel) pre-equilibrated with a 0.1 M NaCl buffer that has been deoxygenated for 30 minutes by sparging with argon.

2 ml of bluish fraction corresponding to an elution from 25 to 27 ml are recovered in a round-bottomed flask. 2 ml of solution of sodium metabisulfite in the NaCl buffer (2.5×10⁻⁴ M) are then added so as to initiate the polymerization, which takes place under an inert atmosphere at 37° C. and for 1 (acrylamide) and 3 (THAM) hours.

In the case of an initiation with sodium dithionite, 2 ml of a solution of the initiator in the 0.1 M NaCl buffer (2×10⁻³ M) are added after separation on a Sephadex column.

### EXAMPLE 8

### Telomerization of a Monomer Encapsulated in Tubular Vesicles Obtained from the Lipid A1

The composition of the solutions used is summarized in Table 2.

TABLE 2

| | Designation | Solvent | Concentration |
|---|---|---|---|
| Monomer | THAM | Distilled water | 0.1 M |
| Telogenic | cholesteryl 3- | Dichloromethane | 2.10⁻³ M |
| agent | mercaptopropanoate | | |
| Initiator | tBuOOH/Na₂S₂O₅ | Dichloromethane/ | 2.5 × 10⁻⁴ M/ |
| | | buffer | 2.5 × 10⁻⁴ M |
| | Na₂S₂O₅ | Buffer | |
| Buffer | NaCl | Distilled water | 0.1 M |

Preparation of the Film

18 mg of lipid A1 and 2 mg of telogenic compound D are solubilized in 2 ml of a solution of cumene hydroperoxide in dichloromethane that has been freshly distilled and degassed by spurging with argon (2.5×10⁻⁴ M), in a heart-shaped round-bottomed flask.

The solution is evaporated to dryness in a rotary evaporator (the bath temperature not exceeding 40° C.) and the film is then dried with a vane pump (1 hour) and placed under an inert atmosphere until use. In the case of an initiation with sodium dithionite, the film is prepared in dichloromethane that has been freshly distilled and degassed by sparging with argon.

Dispersion

2 ml of solution of monomer in distilled water (0.1 M), deoxygenated beforehand by sparging with argon, are added. The mixture is stirred for 1 minute and then placed in an ultrasound bath for 60 minutes at 70° C.

Separation-polymerization

The preparation is loaded on to a Sephadex G50 column (2 cm in diameter for a height of 10 cm of gel) pre-equilibrated with a 0.1 M NaCl buffer that has been deoxygenated for 30 minutes by sparging with argon.

2 ml of bluish fraction corresponding to an elution from 25 to 27 ml are recovered in a round-bottomed flask. 2 ml of solution of sodium metabisulfite in the NaCl buffer (2.5×10⁻⁴ M) are then added so as to initiate the telomerization, which takes place under an inert atmosphere, at 37° C. and for 16 to 20 hours. In the case of an initiation with sodium dithionite, 2 ml of a solution of the initiator in the 0.1 M NaCl buffer (2×10⁻³ M) are added after separation on a Sephadex column.

### EXAMPLE 9

### Synthesis of a Surfactant of General Formula (IB)

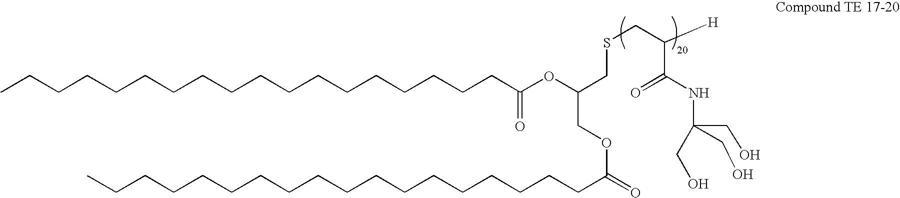 rac 3-(tritylmercapto)propane-1,2-diol (Compound 10)

10 g (92.6 mmol) of 3-mercapto-1,2-propanediol and 13.54 ml (97.23 mmol, 1.05 eq.) of triethylamine (TEA) are solubilized in 100 ml of THF. 27.07 g (97.23 mmol, 1.05 eq.) of triphenylmethyl chloride dissolved in 10 ml of THF are added dropwise to the mixture at a temperature below 30° C. At the end of the addition, the reaction mixture is left in the cold, with stirring, while maintaining the pH at 8-9 by the addition of TEA. The excess triphenylmethyl chloride is eliminated by the addition of a saturated NaHCO₃ solution, before evaporating off the THF under reduced pressure. The crude product is taken up in CH₂Cl₂ before being washed with a normal solution of HCl and then of NaHCO₃, and being dried over Na₂SO₄. The product is finally purified by chromatography on a silica gel column eluted with a gradient (7:3 to 1:1 cyclohexane/EtOAc). 28.8 g of pure product are obtained in the form of a white powder. Rf_{product}: 0.4 (TLC-7:3 EtOAc/cyclohexane). Yield: 89%. Mp: 97-98° C.

¹H NMR(/CDCl₃): δ (ppm) 7.42-7.27 (m, 15H, aromatics from trityl); 3.50 (m, 3H, CHOH, CH₂OH); 2.80 (m, 1H, OH); 2.50 (t, 3H, OH, CH₂S) ¹³C NMR (CDCl₃): δ (ppm) 144.6 (SCC phenyl×3); 129.6 (Cₚₐᵣₐ phenyl×3); 128.0 (Cₒᵣₜₕₒ phenyl×6); 126.8 (Cₘₑₜₐ phenyl×6); 70.6 (CHOH); 67.0 (SCPh₃); 65.5 (CH₂OH); 35.4 (CH₂S).

rac-3-(tritylmercapto)propane-1,2-diyl diheptadecanoate (compound 11)

0.5 g (1.43 mmol) of compound 10 and 0.523 g (4.28 mmol, 3 eq.) of dimethylaminopyridine (DMAP) are solubilized in 10 ml of CH₂Cl₂ and cooled using an ice bath. 0.952 g (3.14 mmol, 2.2 eq.) of stearoyl chloride solubilized in 5 ml of CH₂Cl₂ is then added, dropwise, by means of a dropping funnel. The reaction medium is left for 2 hours, with stirring, after the addition has been completed. The DMAP salts formed are eliminated by filtration and the reaction crude is then evaporated to dryness and finally taken up in a mixture of MeOH/Et₂O from which the product crystallizes. 1.07 g of pure product are obtained in the form of a white powder. Rf_{product}: 0.3 (TLC-7:3 EtOAc/cyclohexane). Yield: 85%. Mp: 52-53° C.

¹H NMR(CDCl₃): δ (ppm) 7.30-7.15 (m, 15H, aromatics from trityl); 5.16 (quint., 1/3H, CHO); 4.78 (quint., 2/3H, CHO); 4.1 (dddd, 2H, CH₂O); 3.10 (t, 2/3H, CH₂S); 2.48 (t, 4/3H, CH₂S); 2.21 (m, 4H, CH₂CO×2); 1.30 (m, 60H, (CH₂)₁₅); 0.88 (t, 6H, CH₃) ¹³C NMR (/CDCl₃): δ (ppm) 172.2 (OCOCH₂×2); 144.4 (SCC phenyl×3); 129.7 (Cₚₐᵣₐ phenyl×3); 128.2 (Cₒᵣₜₕₒ phenyl×6); 127.1 (Cₘₑₜₐ phenyl×6); 70.2 (CHO); 67.4 (SCPh₃); 64.1 (CH₂O); 34.2 (CH₂COO×2); 32.6 (CH₂S); 29.5 ((CH₂)n×2); 24.9 (CH₂CH₂COO×2); 22.7 (CH₂CH₃×2); 14.1 (CH₃×2).

rac-3-Mercaptopropane-1,2-diyl diheptadecanoate (compound 12)

Using 0.5 g (0.56 mmol) of compound 11 and 0.066 g (0.56 mmol, 1 eq.) of triethylsilane, after the addition of a 5% solution of TFA in CH₂Cl₂ and the usual washing, the reaction crude is evaporated to dryness and purified by crystallization from a mixture of Et₂O/MeOH. 0.326 g of pure product is thus recovered in the form of a white powder. Rf_{product}: 0.5 (TLC-7:3 ethyl acetate/cyclohexane). Yield: 90%. Mp: 49-51° C.

hu 1H NMR(/CDCl₃): δ (ppm) 5.1 (m, 1H, CHO); 4.31 (ddd, 2H, CH₂O); 3.10 (t, 2/3H, CH₂S); 2.73 (t, 4/3H, CH₂S); 2.30 (m, 4H, CH₂CO×2); 1.47 (SH); 1.25 (m, 60H, (CH₂)₁₅×2); 0.88 (t, 6H, CH₃×2) ¹³C NMR (/CDCl₃): δ (ppm) 172.5 (CH₂OCO, CHOCO); 71.8 (CHO); 62.4 (CH₂O); 34.2 (CH₂COO×2); 32.4 (CH₂SH); 29.5 ((CH₂)n×2); 24.9 (CH₂CH₂COO×2); 22.7 (CH₂CH₃×2); 14.1 (CH₃×2).

Compound TE 17-20

1.09 g (6.25 mmol, 5 eq.) of tris(hydroxymethyl)acrylamidomethane are solubilized in 15 ml of freshly distilled MeOH. The mixture is stirred and sparged with argon and then heated. As soon as boiling point is reached, a solution containing 0.04 g (0.25 mmol, 0.2 eq.) of AIBN and 0.8 g (1.25 mmol) of Compound 12 in a minimum amount of freshly distilled THF (approximately 1 ml) degassed beforehand with a stream of argon is injected. The reaction is monitored by thin layer chromatography, with disappearance of the thiol (7:3 EtOAc/cyclohexane). After a return to ambient temperature, the reaction crude is immersed in cold Et₂O with vigorous stirring from where the telomer precipitates. 1.2 g of product is obtained in the form of a white powder. Yield: 70%.

¹H NMR (/DMSO): δ (ppm) 7.23 (m, 19.8H, NH×DPN); 4.78 (m, 60H, OH×3×DPn); 0.86 (m, 6H, CH₃×2) DPn:20.

1-22. (canceled)

23. A compound of formula (III): 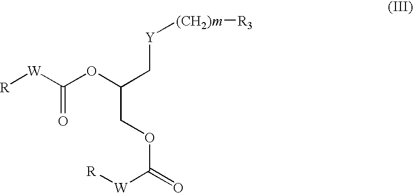
in which R₃ represents a group selected from the group consisting of: 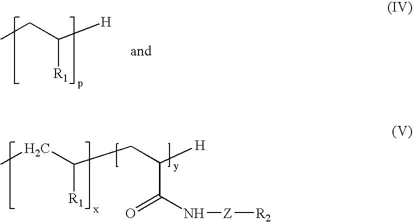
wherein:
Y represents a sulfur atom or an
—NH—CO—(CH₂)n-X group, X represents a sulfur atom or a —CH₂— group; n is an integer ranging from 0 to 10;
R represents a group selected from the group consisting of:
C₄-C₂₄ hydrocarbon-based radicals; and C₄-C₂₄ fluorinated hydrocarbon-based radicals; C₄-C₂₄ thioalkyl radicals;
W represents an —NH— or —CH₂— group;
p represents an integer ranging from 1 to 50;
m is an integer ranging from 0 to 9, and, when X=CH₂, then 0<m+n<6;
x represents an integer ranging from 1 to 30;
y represents 0 or an integer ranging from 1 to 10;
R₁ represents a hydrophilic group;
R₂ represents a recognition group having an affinity for a biological target;
Z is a spacer arm; Z is bound to R₂ by means of a bond which is selected from the group consisting of functions —O—CO—, —CO—NH—, —NH—CO—NH—, —NH—CO—O—, O—CO—O—, —O—, —CH=N—, —S— and by complexation of a nickel atom; Z is selected from the group consisting of a peptide chain, an Ω-amino acid, ethanolamine, 3-propanolamine and a diamine of formula —NH—(CH₂)_{p′}—NH—, in which p′ represents an integer ranging from 2 to 6, or -Z-R₂ represents a group of the formula below:

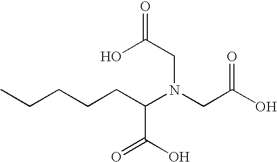

24. The compound of claim 23, wherein the group R is selected from the group consisting of:
thiooctyl radical,
n-butyl, tert-butyl, isobutyl, n-pentyl, isopentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl and the phytyl radical, (CH₃[CH(CH₃)(CH₂)₃]₃CH(CH₃)CH₂CH₂),
fluorinated hydrocarbon-based radicals corresponding to the formula —(CH₂)ₜ— (CF₂)ᵣF, in which r and t represent two integers wherein 14≧r+t≧4.

25. The compound of claim 24, corresponding to formula (I): 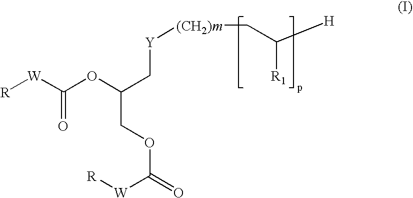
in which:
Y represents a sulfur atom or a group 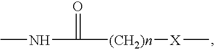
X being selected from the group consisting of S and CH₂ groups, n is an integer ranging from 0 to 10;
m is an integer ranging from 0 to 9; and, when X=CH₂, then 0<m+n<6;
W represents an —NH— group or a —CH₂— group;
p represents an integer ranging from 1 to 50;
R₁ represents a group selected from the group consisting of: 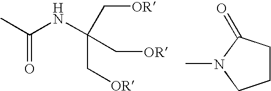
in which R′ represents H or a hydrophilic group;
R represents a group selected from the group consisting of: C₄-C₂₄ hydrocarbon-based radicals; C₄-C₂₄ fluorinated hydrocarbon-based radicals; and C₄-C₂₄ thioalkyl radicals.

26. The compound of claim 25, having formula (IA): 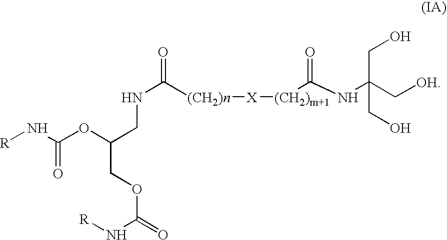
in which:
X represents a sulfur atom or a —CH₂— group;
n is an integer ranging from 0 to 10;
m is an integer ranging from 0 to 9;
when X=CH₂, then 0<m+n<6;
R represents a group selected from the group consisting of:
C₄-C₂₄ hydrocarbon-based radicals; C₄-C₂₄ fluorinated hydrocarbon-based radicals; and C₄-C₂₄ thioalkyl radicals.

27. The compound of claim 26, wherein R is selected such that (IA) has a phase transition temperature of greater than 37° C.

28. The compound of claim 26, having formula A:

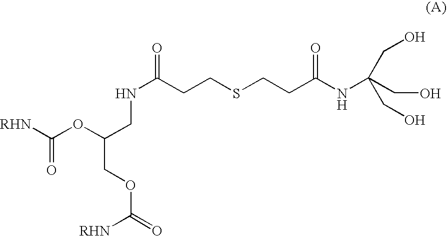

29. The compound of claim 28, having formula A1:

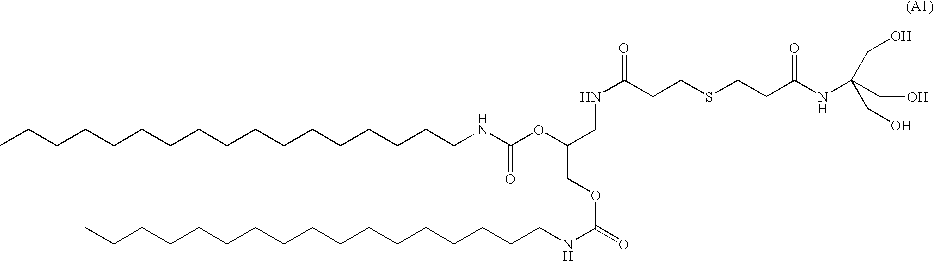

30. The compound of claim 25, having formula (IB): 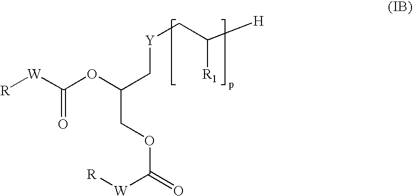
in which:
Y represents a sulfur atom or —NH—CO—CH₂CH₂S— group;
W represents an —NH— group or a —CH₂— group;
p represents an integer ranging from 1 to 50;
R₁ represents a group selected from the group consisting of: 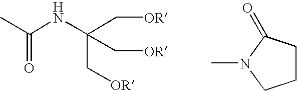
in which R′ represents H or a C₄-C₂₄ polyhydroxylated hydrocarbon-based compound;
R represents a group chosen from: C₄-C₂₄ hydrocarbon-based radicals; C₄-C₂₄ fluorinated hydrocarbon-based radicals; C₄-C₂₄ thioalkyl radicals.

31. The compound of claim 30, wherein R is selected such that (IB) has a critical micellar concentration of less than 10⁻⁵ M.

32. The compound of claim 30, which satisfies one or more of the conditions below:
p represents an integer ranging from 1 to 5;
Y represents a sulfur atom, or both.

33. The compound of claim 30, having formula C, in which p represents an integer ranging from 5 to 15:

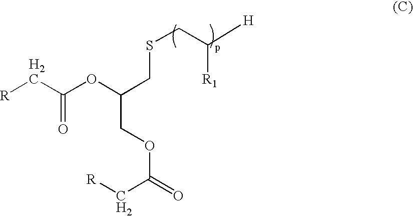

34. The compound of claim 33, having formula C1:

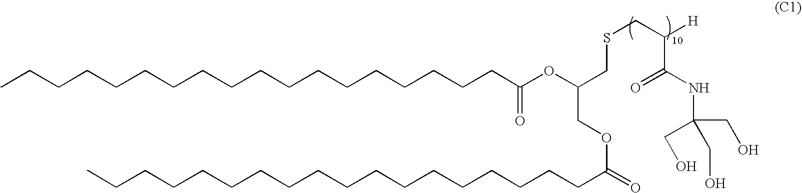

35. The compound of claim 23, having formula (II): 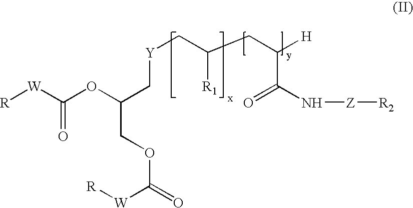
in which:
Y represents a sulfur atom or the —NH—CO—(CH₂)n-X— group, in which X represents a sulfur atom or a —CH₂— group, n is an integer ranging from 0 to 10;
W represents an —NH— or —CH₂— group;
x represents an integer ranging from 1 to 30;
y represents 0 or an integer ranging from 1 to 10;
R₁ represents a hydrophilic group;
R₂ represents a recognition group having an affinity for a biological target;
Z is a spacer arm; Z is bound to R₂ by means of a bond which is selected from the group consisting of functions —O—CO—, —CO—NH—, —NH—CO—NH—, —NH—CO—O—, O—CO—O—, —O—, —CH=N— or —S— or by complexation of a nickel atom; Z is selected from the group consisting of a peptide chain, an Ω-amino acid, ethanolamine, 3-propanolamine and a diamine of formula —NH—(CH₂)_{p′}—NH—, in which p′ represents an integer ranging from 2 to 6, or -Z-R₂ represents a group of formula:

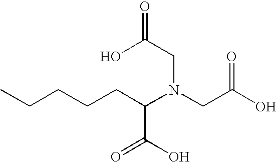

36. The compound of claim 35, having formula (IIA):

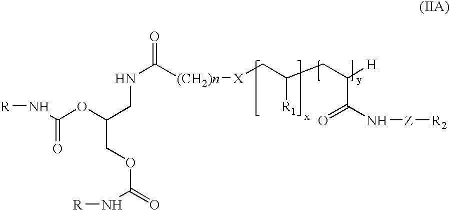

37. The compound of claim 36, wherein one or more of the following conditions are met:
X=S,
n=2,
R₁ is selected from the group consisting of: 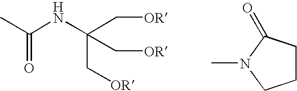
in which R′ represents H or a C₄-C₂₄ polyhydroxylated hydrocarbon-based compound,
R is selected from the group consisting of:
thiooctyl radical,
n-butyl, tert-butyl, isobutyl, n-pentyl, isopentyl, n-hexyl, n-heptyl, n-octyl, n-nonyl, n-decyl, n-undecyl, n-dodecyl, n-tridecyl, n-tetradecyl, or the phytyl radical, (CH₃[CH(CH₃)(CH₂)₃]₃CH(CH₃)CH₂CH₂),
fluorinated hydrocarbon-based radicals corresponding to the formula —(CH₂)ₜ—(CF₂)ᵣF, in which r and t represent two integers wherein 14≧r+t≧4,
R₂ comprises antibodies, antibody fragments, or small effector molecules that allow interaction with cell surface receptors, antigens, sugars and peptides.

38. The compound of claim 37, having formula F:

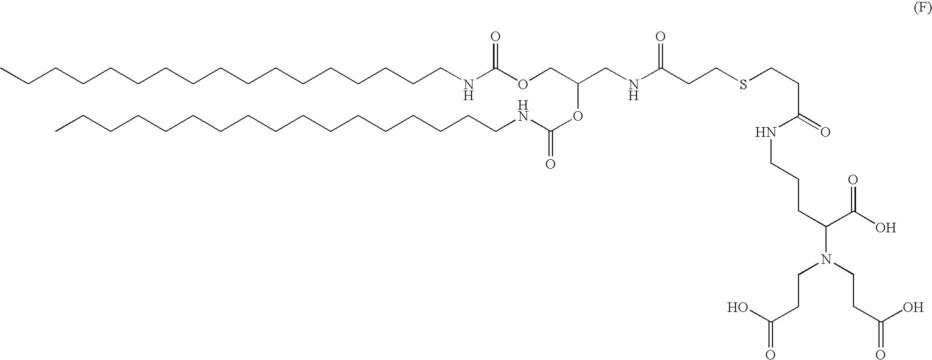

39. The compound of claim 35, having formula (IIB): 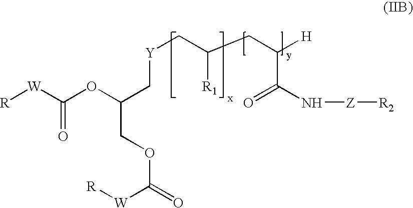
in which:
Y represents a sulfur atom or the —NH—CO—CH₂CH₂S— group.

40. A nanoparticle comprising one or more compounds of formula (I) of claim 25 as a constituent of walls thereof.

41. The nanoparticle of claim 40, which comprises from about 1 to 5% of one or more compounds of formula (II) of claim 35.

42. The nanoparticle of claim 40, which further comprises an acrylic telomer or acrylic polymer in an inner aqueous cavity thereof.

43. A nanoparticle composition comprising nanoparticles of claim 40, and a compound comprising a therapeutic compound, diagnostic compound on a vaccine compound.

44. The nanoparticle composition of claim 43, which is a therapeutic composition comprising nanoparticles and a therapeutic compound.

45. The nanoparticle composition of claim 43, which is a diagnostic composition comprising nanoparticles and a diagnostic compound.

46. The nanoparticle composition of claim 43, which is a vaccine composition comprising nanoparticles and a vaccine compound.
